(19) United States
(12) Patent Application Publication
(10) Pub. No.: US 2025/0353252 A1
(43) Pub. Date: Nov. 20, 2025

(54) METHODS AND SYSTEMS FOR THREE-DIMENSIONAL PRINTING

(71) Applicant: PRELLIS BIOLOGICS, INC., Berkeley, CA (US)

(72) Inventors: Brian HACHTMANN, Berkeley, CA (US); Oleg KONINGS, Berkeley, CA (US); Mackinley KATH, Berkeley, CA (US); Melanie MATHEU, Berkeley, CA (US)

(73) Assignee: PRELLIS BIOLOGICS, INC., Berkeley, CA (US)

(21) Appl. No.: 18/871,132

(22) PCT Filed: May 31, 2023
(86) PCT No.: PCT/US2023/067702

§ 371 Date: Dec. 2, 2024

Related U.S. Application Data
(60) Provisional application No. 63/347,849, filed on Jun. 1, 2022.

(51) Int. Cl. B29C 64/135; B29C 64/171; B29C 64/268; B29C 64/393; B29K 105/04; B29L 31/00; B33Y 10/00; B33Y 50/02; B33Y 70/00; B33Y 80/00

(52) U.S. Cl. CPC B29C 64/135; B29C 64/171; B29C 64/268; B29C 64/393; B33Y 10/00; B33Y 50/02; B33Y 70/00; B33Y 80/00; B29K 2105/04; B29K 2995/0056; B29L 2031/7532

(57) ABSTRACT

Provided herein are methods and systems for improving performance of three-dimensional printing systems, which may include: printing a first portion of the 3D object using a first parameter set and a first light beam, wherein the first parameter set includes at least one first parameter corresponding to a first optical property of the first light beam; and printing a second portion of the 3D In object different from the first portion using a second parameter set and a second light beam, wherein the second parameter set includes at least one second parameter corresponding to a second optical property of the second light beam, wherein the second parameter set is different from the first parameter set, wherein the second optical property is different from the first optical property, to yield at least at least a portion of the 3D object comprising the first portion and the second portion.

### CROSS-REFERENCE

This application claims the benefit of U.S. Provisional Application No. 63/347,849, filed on Jun. 1, 2022, the entirety of which is incorporated herein by reference.

### BACKGROUND

Advances in additive manufacturing, especially three-dimensional (3D) printing, have improved the quality of objects that can be made in short timeframes. Optical 3D printing has improved as well, though limitations and tradeoffs still exist in the speed and resolution that can be achieved.

### SUMMARY

In an aspect, the present disclosure provides a method for printing a three-dimensional (3D) object, comprising: (a) printing a first portion of the 3D object using a first parameter set and a first light beam, wherein the first parameter set includes at least one first parameter corresponding to a first optical property of the first light beam; and (b) printing a second portion of the 3D object different from the first portion using a second parameter set and a second light beam, wherein the second parameter set includes at least one second parameter corresponding to a second optical property of the second light beam, wherein the second parameter set is different from the first parameter set, wherein the second optical property is different from the first optical property, to yield at least at least a portion of the 3D object comprising the first portion and the second portion.

In some embodiments, the first or second parameter set each comprise one or more parameters individually selected from the group consisting of voxel count, mod value, dwell time, illumination time, and optical power. In some embodiments, the first portion and the second portion comprise different feature sizes. In some embodiments, using the first parameter set and the second parameter set reduces an overprinting or an over-curing of the 3D object. In some embodiments, the first light beam and the second light beam are both generated by a same light source. In some embodiments, the light source is a laser light source. In some embodiments, the method further comprises printing a third portion of the 3D object different from the first portion or the second portion using a third parameter set and a third light beam. In some embodiments, the third parameter set comprises a gradient of parameters between the first parameter set and the second parameter set. In some embodiments, the method further comprises printing a second 3D object configured to provide feedback on the printing the first portion and the printing the second portion. In some embodiments, the first portion and the second portion have different properties. In some embodiments, the properties are selected from the group consisting of feature size, tensile strength, porosity, Young's modulus, yield strength, degradation rate, swelling properties, protein composition, and polymer composition. In some embodiments, the 3D object comprises one or more biopolymers. In some embodiments, the using the second parameter set for the printing the second portion reduces an overcuring of the second portion as compared to using the first parameter set to print the second portion. In some embodiments, the printing the 3D object comprises printing a plurality of portions to form the 3D object, wherein the plurality of portions comprises the first portion and the second portion. In some embodiments, the first parameter set is configured to achieve a first predetermined level of cure of the first portion, and wherein the second parameter set is configured to achieve a second predetermined level of cure of the second portion. In some embodiments, the second portion is at least partially disposed within the first portion, or vice versa In some embodiments, the second portion is disposed within the first portion, or vice versa In some embodiments, the 3D object is printed at a smaller size than a size where the 3D object will be used. In some embodiments, the 3D object is printed 5% smaller than the size where the 3D object will be used. In some embodiments, the 3D object is exposed to agents configured to swell the 3D object to the size where the 3D object will be used. In some embodiments, the agents comprise phosphate buffered saline. In some embodiments, the first portion and the second portion are printed at a substantially same time. In some embodiments, the 3D object is printed in a time period of at most about 6 hours. In some embodiments, the 3D object comprises at least one cell. In some embodiments, the at least one cell is of a subject. In some embodiments, the at least one cell is present in a media chamber prior to the directing. In some embodiments, the at least one cell is introduced to the 3D object subsequent to generating the object. In some embodiments, the first light beam comprises a holographic projection of the first portion or the second portion. In some embodiments, the light beam comprises a plurality of energy beams. In some embodiments, the 3D object corresponds to an organ or organoid selected from the group consisting of a two-dimensional organ or organoid, a three-dimensional organ or organoid, a lymph node, an islet of Langerhans, a hair follicle, a tumor or a tumor spheroid, a neural bundle and support cell(s), a nephron, a liver organoid, an intestinal crypt, a primary lymphoid organ, a secondary lymphoid organ, a spleen, a liver, a pancreas, a gallbladder, an appendix, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and a blood vessel. In some embodiments, the method further comprises receiving computer instructions for printing the 3D object, and forming at least the first portion or the second portion based at least in part on the computer instructions. The method of claim 28, wherein the computer instructions comprise a computer model of the object. In some embodiments, the 3D object comprises a polymeric material, a metal, a metal alloy, a composite material, or any combination thereof. In some embodiments, the light beam is phase modulated. In some embodiments, the 3D object comprises signaling molecules or proteins. In some embodiments, the method further comprises, subsequent to (a), developing the 3D object into a biologically functional tissue. In some embodiments, the light beam is generated by least one laser source. In some embodiments, the laser source is a two-photon energy source.

In another aspect, the present disclosure provides a method of generating a computer file corresponding to a three-dimensional (3D) object, wherein the computer file is usable for printing the 3D object using a three-dimensional (3D) printer, the method comprising: (a) receiving a computer model of the 3D object into computer memory; (b) slicing the computer model to form a plurality of voxels; (c) distributing the plurality of voxels into a plurality of constellations, wherein a constellation of the plurality of constellations comprises at least one voxel of the plurality of voxels, wherein the constellation of the plurality of constellations and another constellation of the plurality of constellations are curable with an approximately same optical power; and (d) generating the computer file comprising the plurality of constellations.

In some embodiments, the plurality of voxels are oriented in three dimensions relative to one another.

In another aspect, the present disclosure provides a method of preparing a file corresponding to a three-dimensional (3D) object for printing using a three-dimensional (3D) printer, comprising: (a) receiving a plurality of clusters generated by a k-means fracturing algorithm; and (b) recombining the plurality of clusters by maximizing a centroid distance for each cluster of the plurality of clusters.

In another aspect, the present disclosure provides a method of printing a three-dimensional (3D) object using a three-dimensional (3D) printer, comprising: (a) using the 3D printer to cure a first portion of the 3D object; and (b) using the 3D printer to cure a second portion of the 3D object, wherein the first portion and the second portion form an at least partially overlapping area, and wherein the at least partially overlapping area has a substantially same level of cure as the first portion and the second portion.

In another aspect, the present disclosure provides a method of printing a three-dimensional (3D) object using a three-dimensional (3D) printer, comprising: (a) using the 3D printer to provide a first patterned light field to cure a first portion of the 3D object; and (b) using the 3D printer to provide a second patterned light field to cure a second portion of the 3D object at least partially overlapping with the first portion of the 3D object to form an at least partially overlapping portion, wherein the first patterned light field and the second patterned light field comprise a region of lower light intensity within the at least partially overlapping portion.

In another aspect, the present disclosure provides a method of troubleshooting a three-dimensional (3D) printing process using a 3D printer, comprising: (a) using the 3D printer to print a first object; (b) using the 3D printer to print a second object, wherein the second object comprises a circle with an equilateral cross disposed therein; and (c) comparing the second object with a computer file for the second object to troubleshoot the 3D printing process.

In some embodiments, the first object or the second object is printed in a time period of at most about 6 hours. In some embodiments, the first object and the second object are printed at a substantially same time. In some embodiments, the first object comprises at least one cell. In some embodiments, the at least one cell is of a subject. In some embodiments, the at least one cell is present in the media chamber prior to the printing. In some embodiments, the at least one cell is introduced to the object subsequent to the printing. In some embodiments, the printing comprises directing a three-dimensional holographic projection of at least one energy beam into a media chamber. In some embodiments, the first object corresponds to an organ or organoid selected from the group consisting of a two-dimensional organ or organoid, a three-dimensional organ or organoid, a lymph node, an islet of Langerhans, a hair follicle, a tumor or a tumor spheroid, a neural bundle and support cell(s), a nephron, a liver organoid, an intestinal crypt, a primary lymphoid organ, a secondary lymphoid organ, a spleen, a liver, a pancreas, a gallbladder, an appendix, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and a blood vessel. In some embodiments, the method further comprises receiving computer instructions for printing the first object or the second object, and forming at least the portion of the first object or the second object based at least in part on the computer instructions. In some embodiments, the computer instructions comprise a computer model of the first object or the second object. In some embodiments, the first object or the second object comprises a polymeric material, a metal, a metal alloy, a composite material, or any combination thereof. In some embodiments, the first object comprises signaling molecules or proteins. In some embodiments, the method further comprises, subsequent to (a), developing the first object into a biologically functional tissue.

In another aspect, the present disclosure provides a method of troubleshooting a three-dimensional (3D) printing process using a 3D printer, comprising: (a) using the 3D printer to print a first object; (b) using the 3D printer to print a second object, wherein the second object comprises a plurality of cross-hatched lattices and wherein a distance between the lattices is asymmetrical; and (c) comparing the second object with a computer file for the second object to troubleshoot the 3D printing process

In some embodiments, the first object and the second object are printed at a substantially same time. In some embodiments, the first object or the second object is printed in a time period of at most about 6 hours. In some embodiments, the first object or the second object comprises at least one cell. In some embodiments, the at least one cell is of a subject. In some embodiments, the at least one cell is present in prior to the printing. In some embodiments, the at least one cell is introduced to the first object or the second object subsequent to generating the first object or the second object. In some embodiments, the printing comprises directing a three-dimensional holographic projection of at least one energy beam into a media chamber. In some embodiments, the first object corresponds to an organ or organoid selected from the group consisting of a two-dimensional organ or organoid, a three-dimensional organ or organoid, a lymph node, an islet of Langerhans, a hair follicle, a tumor or a tumor spheroid, a neural bundle and support cell(s), a nephron, a liver organoid, an intestinal crypt, a primary lymphoid organ, a secondary lymphoid organ, a spleen, a liver, a pancreas, a gallbladder, an appendix, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and a blood vessel. In some embodiments, the method further comprises receiving computer instructions for printing the first object or the second object, and forming at least the portion of the first object or the second object based at least in part on the computer instructions. In some embodiments, the computer instructions comprise a computer model of the first object or the second object. In some embodiments, the first object or the second object comprises a polymeric material, a metal, a metal alloy, a composite material, or any combination thereof. In some embodiments, the first object comprises signaling molecules or proteins. In some embodiments, the method further comprises, subsequent to (a), developing the first object into a biologically functional tissue.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a 3D object comprising a first portion and a second portion, according to some embodiments.

FIG. 2 is an example of an 3D object comprising four different portions, according to some embodiments.

FIG. 3 is a microscope image of an example of a 3D object, according to some embodiments.

FIGS. 4A-4C are examples of a constellation formation process, according to some embodiments.

FIG. 5 shows an example of voxel partitioning, according to some embodiments.

FIGS. 6A-6B show examples of voxel stitching, according to some embodiments.

FIG. 7 shows examples of light profiles to reduce overcuring in an overlapping fit configuration, according to some embodiments.

FIG. 8A shows a top and isometric view of an object configured for use as a printing diagnostic, according to some embodiments.

FIG. 8B shows an example of a use of an object for a diagnostic during a printing operation, according to some embodiments.

FIG. 9A shows a top view of an object configured for use as a printing diagnostic, according to some embodiments.

FIG. 9B shows an example of a use of an object for a diagnostic during a printing operation, according to some embodiments.

FIG. 10A shows a plurality of objects configured as a diagnostic object, according to some embodiments.

FIG. 10B shows a diagnostic object comprising a plurality of objects in use next to a 3D object, according to some embodiments.

FIG. 11 is an example of a beam blocker, according to some embodiments.

FIG. 12 is an example of a level of cure monitoring configuration for a 3D printer, according to some embodiments.

FIGS. 13A-13D are examples of models of a microfluidics platform, according to some embodiments.

FIG. 14 is an example of microscope images of a microfluidics platform, according to some embodiments.

FIG. 15 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art.

Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

In some cases, a voxel can be a three-dimensional (3D) volume. In some cases, a voxel is an addressable volume of a 3D printer. A voxel may be a unit volume a 3D projection. For example, a voxel can be the smallest building block of a 3D object. A voxel may have a volume of at least about 0.1, 0.5, 1, 2, 3, 4, 5, 6, 7, 8, 9, 10, 50, 100, 500, 1,000, or more cubic micrometers. A voxel may have a volume of at most about 1,000, 500, 100, 50, 10, 9, 8, 7, 6, 5, 4, 3, 2, 1, 0.5, 0.1, or less cubic micrometers.

In another aspect, the present disclosure provides a method for printing a three-dimensional (3D) object. The method may comprise printing a first portion of the 3D object using a first parameter set and a first light beam. The first parameter sent may include at least one first parameter corresponding to a first optical property of the first light beam. A second portion of the 3D object may be printed different from the first portion using a second parameter set and a second light beam. The second parameter set may include at least one second parameter corresponding to a second optical property of the second light beam. The second parameter set may be different from the first parameter set. The second optical property may be different from the first parameter set. The second optical property may be different from the first optical property. At least a portion of the 3D object comprising the first portion and the second portion may be yielded.

The first and/or second parameter set may each independently comprise one or more parameters individually selected from voxel count, mod value, dwell time, illumination time, optical power, or the like, or any combination thereof. The voxel count may be a number of voxels that can be simultaneously printed by a 3D printer. For example, a 3D printing system capable of projecting 1,000 simultaneous voxels can have a voxel count of 1,000. The voxel count may vary depending on the resolution that the object is printed at. The mod value may be an axial or optical axis dimension of a voxel. For example, the mod value may be the z-axis resolution of a voxel for a system that projects light along the z-axis. The mod value may be a distance between printed vertical pixels. The mod value may be less than a resolution of the system in a plane perpendicular to the axis or optical axis (e.g., an xy plane). For example, for a system with a xy resolution of 1 micrometer can have a mod value of 5 micrometers. A 3D printing system can have a resolution or mod value of at least about 10 nanometers (nm), 50 nm, 100 nm, 200 nm, 300 nm, 400 nm, 500 nm, 600 nm, 700 nm, 800 nm, 900 nm, 1 micrometer (μm), 2 μm, 3 μm, 4 μm, 5 μm, 6 μm, 7 μm, 8 μm, 9 μm, 10 μm, 25 μm, 50 μm, 75 μm, 100 μm, 250 μm, 500 μm, 1,000 μm, 5,000 μm, 10,000 μm, or more. A 3D printing system can have a resolution or mod value of at most about 10,000 μm, 5,000 μm, 1,000 μm, 500 μm, 100 μm, 75 μm, 50 μm, 25 μm, 10 μm, 9 μm, 8 μm, 7 μm, 6 μm, 5 μm, 4 μm 3 μm, 2 μm, 1 μm, 900 nm, 800 nm, 700 nm, 600 nm, 500 nm, 400 nm, 300 nm, 200 nm, 100 nm, 50 nm, 10 nm, or less. A 3D printing system can have a resolution or mod value in a range as defined by any two of the proceeding values.

The dwell time may be an exposure time of a medium to the light from an optical 3D printer. For example, the dwell time can be the length of time that the media is cured during a particular printing operation. In this example, a plurality of voxels can be exposed to the curing light for a duration of the dwell time. The dwell time may be at least about 0.0000001, 0.0000005, 0.000001, 0.000005, 0.00001, 0.00005, 0.0001, 0.0005, 0.001, 0.005, 0.01, 0.05, 0.1, 0.5, or more seconds. The dwell time may be at most about 0.5, 0.1, 0.05, 0.01, 0.005, 0.001, 0.0005, 0.0001, 0.00005, 0.00001, 0.000005, 0.000001, 0.0000005, 0.0000001, or less seconds. The optical power may be a power delivered via light. The optical power that the system can provide to a media bath may be dependent on the number of voxels the system is configured to generate. For example, the optical power may be the optical power of a laser in the system divided among the voxels of the system. The optical power may be at least about 1, 5, 10, 50, 100, 500, 1,000, 5,000, 10,000, 50,000, 100,000, 500,000, 1,000,000, or more microjoules. The optical power system may have a power of at most about 1,000,000, 500,000, 100,000, 50,000, 10,000, 5,000, 1,000, 500, 100, 50, 10, 5, 1, or less microjoules.

The first portion and the second portion may comprise different feature sizes. For example, the first portion can comprise large features while the second portion comprises smaller features. In this example, the first portion may be printed at a higher optical power to improve printing speed while the second portion may be printed at a lower optical power to improve resolution and feature definition. The using the first parameter set and the second parameter set may reduce an overprinting and/or an over-curing of the 3D object. For example, a smaller feature of the 3D object may have an improved print quality when a parameter set with a lower optical power is used for printing the features. In this example, the lower optical power can reduce over-curing of the small feature. The first light beam and the second light beam may both be generated by a same light source. For example, the light source can generate a single light beam that is split into the first and second light beams. Use of a single light source can reduce system cost and complexity while providing the various benefits described herein. The single light source may comprise a laser, a lamp, a light-emitting diode (LED), a broadband light source with or without a spectrally selective filter, or the like.

The method may further comprise printing a third portion of the 3D object different from the first portion or the second portion using a third parameter set and a third light beam. For example, the third portion can be a portion of a size intermediate between that of the first portion and that of the second portion. The third parameter set may comprise a gradient of parameters between the first parameter set and the second parameter set. For example, a parameter of the first parameter set can have a value of 10, a parameter of the second parameter set can have a value of 1, and the third parameter set can have values at different areas of the third portion of 9, 8, 7, 6, 5, 4, 3, and 2. In another example, the third parameter set can have a value of 5.

The method may comprise printing a second 3D object configured to provide feedback on the printing of the first portion and the printing of the second portion. For example, the second 3D object can be a target as described elsewhere herein (e.g., a gradient target, a cross target, etc.). The second 3D object can be used to diagnose printing issues in the first 3D object without affecting the properties of the first 3D object, improving the consistency of the 3D printing process and the overall quality of the process.

The first portion and the second portion may have different properties. Examples of properties include, but are not limited to, feature size, tensile strength, porosity, Young's modulus, yield strength, degradation rate, swelling properties, protein composition, polymer composition, or the like, or any combination thereof. For example, a first portion can have a first protein composition configured to not bind to a biomolecule, while a second portion can have a second protein composition configured to bind to the biomolecule.

The 3D object may comprise one or more biopolymers. A biopolymer may be a polymer generated by a living organism. Examples of biopolymers may include, but are not limited to, proteins (e.g., polypeptides), nucleic acids (e.g., polynucleotides, deoxyribonucleic acid, ribonucleic acid, etc.), polysaccharides (e.g., carbohydrates, etc.), or the like. The biopolymers may be present in a media bath prior to the formation of the first or second portion of the 3D object. For example, a media bath can comprise monomers and biopolymers. The biopolymers can be introduced to the 3D object subsequent to formation of the 3D object. For example, the biopolymers can be flowed into the 3D object after the 3D object is formed.

The using the second parameter set for the printing the second portion may reduce an overcuring of the second portion as compared to using the first parameter set to print the second portion. For example, for a first portion with a larger feature size than the second portion, the power used to cure the first portion can be higher than the power used to cure the second portion. In this example, using the higher power to cure the second portion can result in overprinting (e.g., curing a larger volume than the size of the second portion), which can result in decreased resolution and object fidelity. In this example, using a lower power for the second portion can decrease overprinting in the second portion while maintaining fast print speeds in the first portion by using a higher power for the first portion. The first parameter set can be configured to achieve a first predetermined level of cure of the first portion. The second parameter set can be configured to achieve a second predetermined level of cure of the second portion. For example, the first and second parameter sets can comprise a combination of optical power, dwell time, number of repetitions, and voxel size to provide a predetermined level of cure for the first and second portions, respectively.

The printing the 3D object may comprise printing a plurality of portions to form the 3D object. The plurality of portions may comprise the first portion and the second portion. The plurality of portions may comprise at least about 2, 3, 4, 5, 6, 7, 8, 9, 10, 15, 25, 50, 75, 100, 250, 500, 750, 1,000, 5,000, 10,000, or more portions. The plurality of portions may comprise at most about 10,000, 5,000, 1,000, 750, 500, 250, 100, 75, 50, 25, 15, 10, 9, 8, 7, 6, 5, 4, 3, or fewer portions. The number of portions in the plurality of portions may be determined at least in part by the properties of each portion of the plurality of portions. For example, for an object with three different regions with different feature sizes can have three different portions. In another example, an object with three regions with different feature sizes and five different functionalized biomolecules can have 15 portions.

The second portions may be at least partially disposed within the first portion. The first portion may be disposed at least partially with the second portion. For example, the first portion can be a region around a cellular niche within a larger second portion. The first portion may be at least partially disposed adjacent to the second portion, or vice versa. The first portion may be at least partially in contact with the second portion. For example, the first portion can be a portion affixed to a side of the second portion. The first portion may be at least about 1, 5, 10, 15, 20, 25, 30, 35, 40, 45, 50, 55, 60, 65, 70, 75, 80, 85, 90, 95, 96, 97, 98, 99, or more percent disposed within the second portion, or vice versa. The first portion may be at most about 99, 98, 97, 96, 95, 90, 85, 80, 75, 70, 65, 60, 55, 50, 45, 40, 35, 30, 25, 20, 15, 10, 5, 1, or less percent disposed within the second portion, or vice versa. The first portion may be totally disposed within (e.g., surrounded by) the second portion, or vice versa.

The 3D object may be printed at a smaller size from a predetermined size of the 3D object (e.g., a size for when the 3D object is in use). For example, a 3D object configured to fill a 10 cm gap can be printed at 9.5 cm. The 3D object may be printed at a smaller size to account for a swelling of the 3D object (e.g., a solvent based swelling). The 3D object may be printed at least about 0.01, 0.05, 0.1, 0.5, 1, 2, 3, 4, 5, 6, 7, 8, 9, 10, 11, 12, 13, 14, 15, 16, 17, 18, 19, 20, or more percent smaller than a predetermined size of the 3D object. The 3D object may be printed at most about 20, 19, 18, 17, 16, 15, 14, 13, 12, 11, 10, 9, 8, 7, 6, 5, 4, 3, 2, 1, 0.5, 0.1, 0.05, 0.01, or fewer percent smaller than a predetermined size of the 3D object. The 3D object can be exposed to agents configured to increase the size of the 3D object to the predetermined size. For example, the 3D object can be printed, cleaned, and exposed to agents to increase the size of the 3D object. In some cases, the agents are additionally used as buffers, solvents, media, or the like. For example, the agents can be a buffer solution for cells growing in the 3D object as well as an agent configured to increase the size of the 3D object.

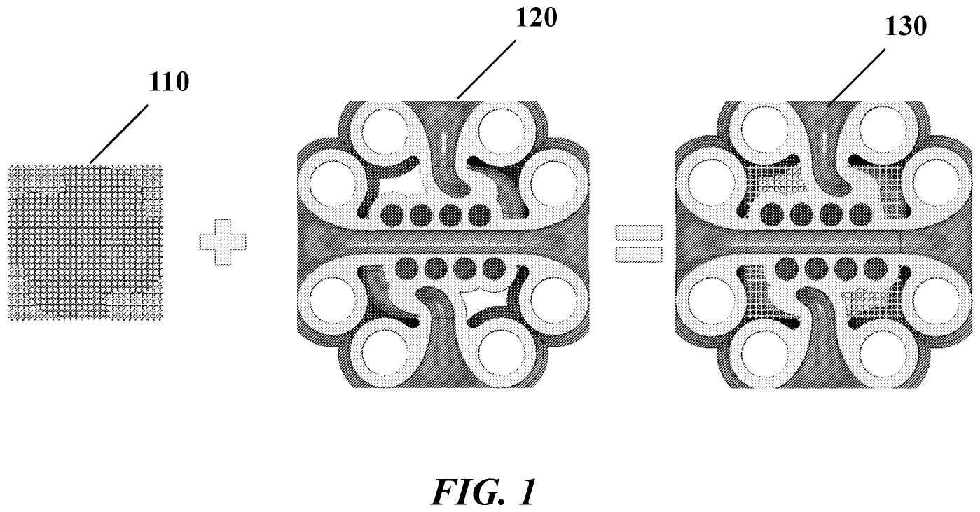

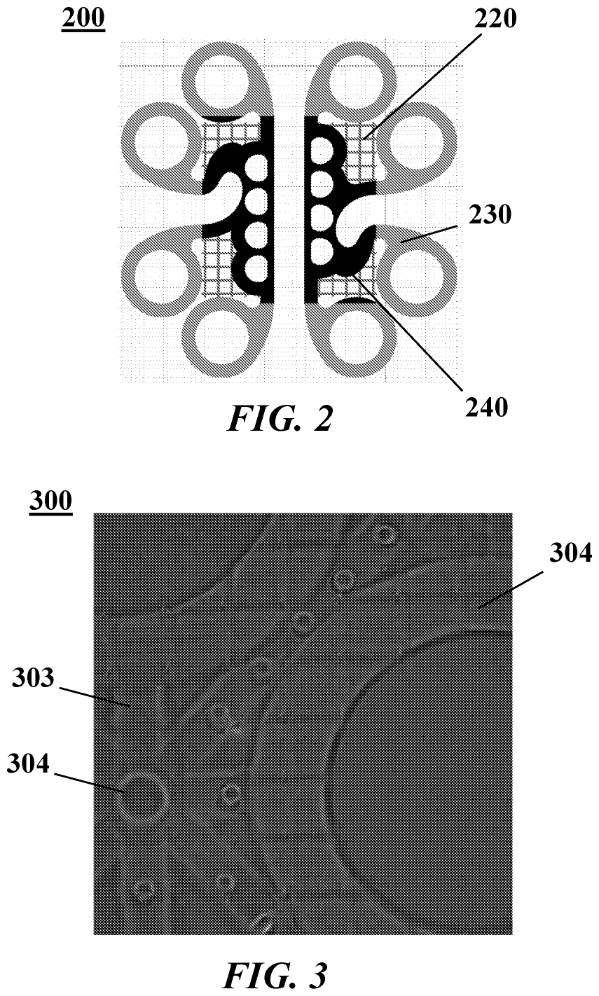

FIG. 1 shows a 3D object 100 comprising a first portion 110 and a second portion 120, according to some embodiments. The first and second portions may comprise different feature sizes. For example, the first portion can comprise a plurality of small features, while the second portions can comprise a plurality of large features. The first portion may be printed with a smaller voxel with lower power and shorter dwell times as compared to the second portion, where larger voxels and higher powers can increase a printing speed. Using the parameters for printing the second portion while printing the first portion can result in the first portion losing resolution, being overcured, and not performing to specification. As such, the 3D object can be printed using a plurality of (e.g., two) different parameter sets. A first parameter set can be configured for the printing of the first portion, for example, by using more voxels, lower mod value, shorter dwell time, shorter illumination time, and lower optical power than a second parameter set configured for the printing of the second portion. FIG. 2 is an example of an 3D object 200 comprising four different portions, according to some embodiments. 3D object 200 can comprise a first portion 210 (e.g., a small lattice), a second portion 220 (e.g., a large lattice), a third portion 230 (e.g., a first large portion with a first predetermined material property), and a fourth portion 340 (e.g., a second large portion with a second predetermined material property. In this example, the third and fourth portions may have a same feature size and voxel count, but the material properties (e.g., hardness, level of cure, etc.) may be different between the portions. In the example of FIG. 2, the different portions can each have a different combination of feature sizes, material properties, etc. where use of a plurality of parameter sets can provide for an improved final 3D object as compared to using a single parameter set for the entire 3D object.

In another aspect, the present disclosure provides a method of generating a computer file corresponding to a three-dimensional (3D) object. The computer file may be usable for printing the 3D object using a 3D printer. A computer model of the 3D object may be received into computer memory. The computer model may be sliced to form a plurality of voxels. The plurality of voxels may be distributed into a plurality of constellations. A constellation of the plurality of constellations may comprise at least one voxel of the plurality of voxels. The constellation of the plurality of constellations and another constellation of the plurality of constellations may be curable with an approximately same optical power. The computer file may be generated comprising the plurality of constellations.

The constellation can be curable with at least about 50, 55, 60, 65, 70, 75, 80, 85, 90, 95, 96, 97, 98, 99, 100, 101, 102, 103, 104, 105, 110, 115, 120, 125, 130, 135, 140, 145, 150, or more percent of an amount of power to cure the another constellation. The constellation can be curable with at most about 150, 145, 140, 135, 130, 125, 120, 115, 110, 105, 104, 103, 102, 101, 100, 99, 98, 97, 96, 95, 90, 85, 80, 75, 70, 65, 60, 55, 50, or less percent of an amount of power to cure the another constellation. The constellation can be curable with an amount of power to cure the another constellation in a range as defined by any two of the proceeding values. For example, the constellation can be cured with an amount of power from 95% to 105% of the power to cure the another constellation. For example, the constellation can comprise a large number of fine details found throughout the 3D object and the another constellation can comprise a small number of large portions of the 3D object. In this example, the total amount of optical power to cure the numerous fine details can be approximately the same as the total amount of optical power to cure the lower number of large portions.

The plurality of voxels may be oriented in three dimensions relative to one another. For example, the plurality of voxels can be positioned such that the plurality of voxels are not in a single plane. The plurality of voxels may be disposed adjacent to one another. For example, the plurality of voxels may be disposed to form a continuous projection in 3D space. The plurality of voxels may be disposed in a non-adjacent manner. For example, the plurality of voxels can be disposed such that the plurality of voxels for at least two projections in 3D space. The plurality of voxels can be disposed such that none of the voxels of the plurality of voxels touch one another.

In some cases, the use of constellations can improve a printing speed of the 3D object by reducing the number of stage movements of the 3D printer. The movement of the stage can be a slow operation as compared to the projection of light in the 3D printer, so recuing the number of stage movements by maximizing the use of the optical power available to the 3D printer can improve the printing time and overall efficiency of the 3D printer. In some cases, where the voxels of the constellations are oriented in three dimensions relative to one another, different parts of the 3D object can be formed at a same time, which can enable higher efficiency printing of the 3D object. For example, the ability to access all three dimensions when forming a constellation, and using non-contiguous voxels for the constellation, can permit high (e.g., optimal) use of the optical power supplied by the 3D printer.

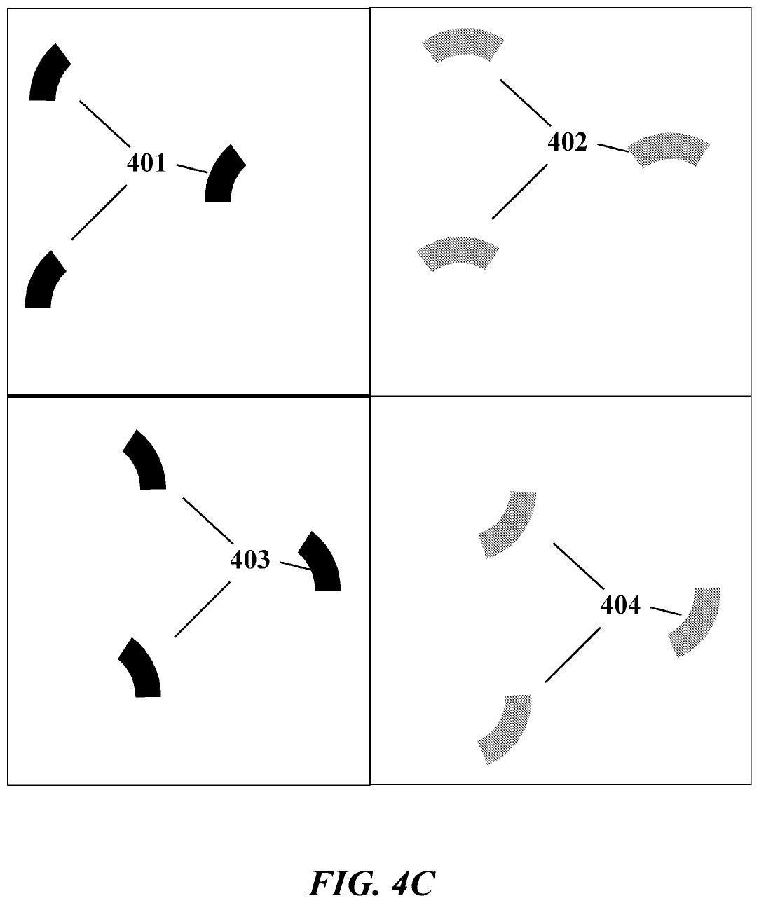

FIGS. 4A-4C are examples of a constellation formation process, according to some embodiments. A plurality of objects 410 can be provided as a computer file to be printed on a 3D printer. The file can be processed to generate instructions for the 3D printer, and the processing can comprise splitting the plurality of objects into a plurality of portions. For example, each object from FIG. 4A can be split into a plurality of portions 420 in FIG. 4B. In this example, the portions may be offset in color for visual clarity. The plurality of portions may form the entirety of the plurality of objects (e.g., the plurality of portions may be sufficient to form the plurality of objects). In FIG. 4C, the different portions can be printed as constellations in the 3D printer. For example, a first constellation can comprise portions 401, while a second constellation can comprise portions 402, a third constellation can comprise portions 403, and a fourth constellation can comprise portions 404. Additional constellations comprising additional portions may not be shown, but can be generated according to the methods described elsewhere herein. The constellations printed at a same time can be separated from one another to reduce printing errors related to too high of photon flux in a given area. For example, the constellations can be printed as shown in FIG. 4C to reduce overcuring of adjacent constellations.

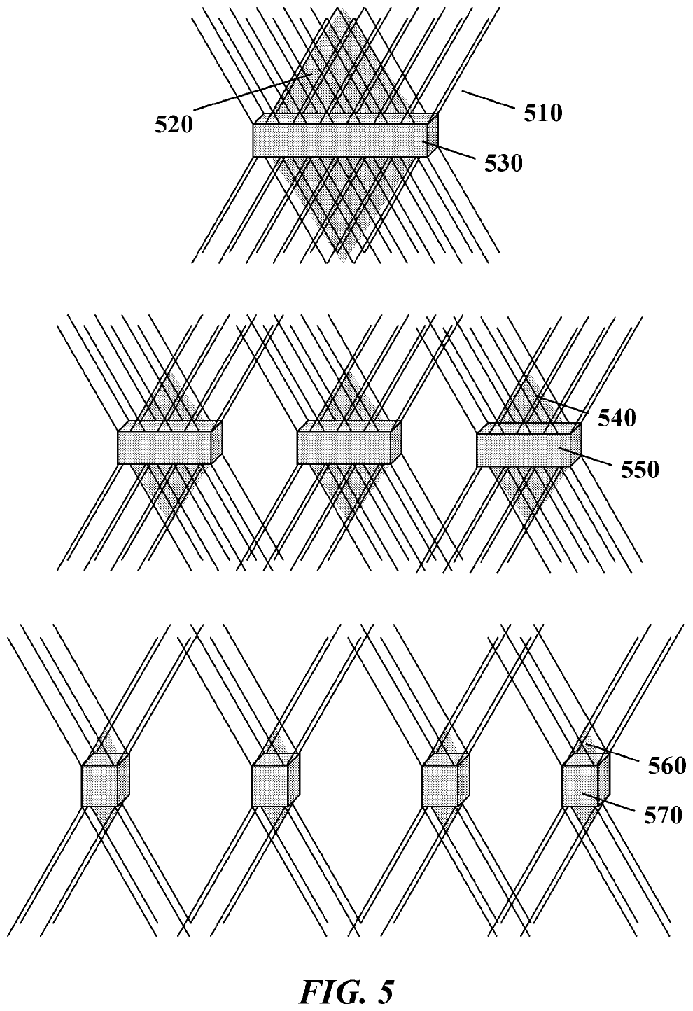

FIG. 5 shows an example of voxel partitioning, according to some embodiments. A light beam projected via a 3D printer 510 can be configured to form a voxel 530. The light can be structured such that the light density is sufficient in the volume of the voxel to cure a medium in the 3D printer. Due to the size of the voxel and the amount of light used to cure it, a region of high light density 520 can be formed above and below the voxel. This region can have a light density sufficient to cause at least partial curing of the media outside of the predetermined voxel. Such curing can result in unintended objects being formed, which can compromise the overall quality of a 3D object. To reduce the amount of this unintended curing, the voxel 530 can instead be separated into smaller voxels 550. By using smaller voxels, the size of the overlapping region 540 can be reduced while maintaining the overall volume of the voxel. The smaller voxels can be printed at different time or in different regions within a larger 3D object to maintain the design and material properties of the overall object while decreasing undesired overcuring. A further shrinking of the voxels to voxels 570 can provide a minimal amount of overcuring in regions 560. The small voxels can then similarly be printed at different times or in different regions of a larger 3D object. The small voxels can be separated by maximal distances to ensure reduced overprinting and reduced correlated printing errors (e.g., overprinting, overheating, etc.).

In another aspect, the present disclosure provides a method of preparing a file corresponding to a 3D object for printing using a 3D printer. A plurality of clusters generated by a k-means fracturing algorithm can be received. The plurality of clusters can be recombined by maximizing a centroid distance for each cluster of the plurality of clusters. By recombining clusters generated by a clustering (e.g., k-means clustering) algorithm based on properties as described elsewhere herein (e.g., cluster size, optical power to print the cluster, etc.), a reduced number of clusters can be yielded that reduces print times while maintaining the properties of the 3D object.

The recombining of the plurality of clusters can be performed to maximize centroid distance between the clusters. Maximizing the centroid distance can result in a scatter or shotgun constellation, where the clusters are printed at a same time as distant clusters. This can reduce local effects created by the printing process (e.g., heating, over polymerization, high radical concentrations, etc.) while permitting printing of multiple clusters at a same time, which can in turn improve the quality of the 3D object.

The recombining the plurality of clusters can comprise use of one or more randomly or pseudo-randomly generated centroids. The distance to the generated centroids can then be calculated for each cluster, the position of the centroids can be adjusted, and the process iterated. The process can be terminated and the final configuration of the clusters can be determined for example, when an error or distance of the clusters to the centroids is the same before and after the iteration. Additionally, the clusters can be moved in the groups until an error is reduced.

In another aspect, the present disclosure provides a method of printing a 3D object using a 3D printer. The 3D printer may be used to cure a first portion of the 3D object. The 3D printer may be used to cure a second portion of the 3D object. The first portion and the second portion may form an at least partially overlapping area. The at least partially overlapping area may have a substantially same level of cure as the first portion and the second portion.

In another aspect, the present disclosure provides a method of printing a 3D object using a 3D printer. The 3D printer may be used to provide a first patterned light field to cure a first portion of the 3D object. The 3D printer may be used to provide a second patterned light field to cure a second portion of the 3D object at least partially overlapping with the first portion of the 3D object to form an at least partially overlapping portion. The first patterned light field and second patterned light field may comprise a region of lower light intensity within the at least partially overlapping portion.

When printing a 3D object in portions, the interface between the portions can be a weak point in terms of mechanical properties as well as adhesion. For example, two portions of a 3D object printed adjacent to one another without overlap can slide apart and destroy the 3D object.

Additionally, simply overlapping the light projections for different parts can result in overcuring of the overlapped portion, which can impart brittleness and reduce the uniformity of the 3D object. If instead the light projection is adjusted such that the edges of the projection have a lower light intensity than the main portion of the projection, the overlap of the two portions can be cured to a substantially same level of cure as one another. The overlap can be cured such that the two portions are within at least about 50, 55, 60, 65, 70, 75, 80, 85, 90, 95, 96, 97, 98, 99, 100, 101, 102, 103, 104, 105, 110, 115, 120, 125, 130, 135, 140, 145, 150, or more percent of the level of cure of the overlap. The overlap can be cured such that the two portions are within at most about 150, 145, 140, 135, 130, 125, 120, 115, 110, 105, 104, 103, 102, 101, 100, 99, 98, 97, 96, 95, 90, 85, 80, 75, 70, 65, 60, 55, 50, or less percent of the level of cure of the overlap.

In some cases, the first portion and the second portion can be printed at a same or substantially same time. For example, the first and second portions can both be printed during a same print operation even though they are printed as parts of different voxels. This can be accomplished by use of a 3D projection of light as described elsewhere herein. In some cases, the first object and the second object are printed at different times. For example, the first portion can be printed, with the boundary between the first portion and the second portion can be partially cured, and subsequently the second portion can be cured. In this example, the boundary region between the first portion and the second portion can be cured to a same level as a result of being exposed to the first and second printings.

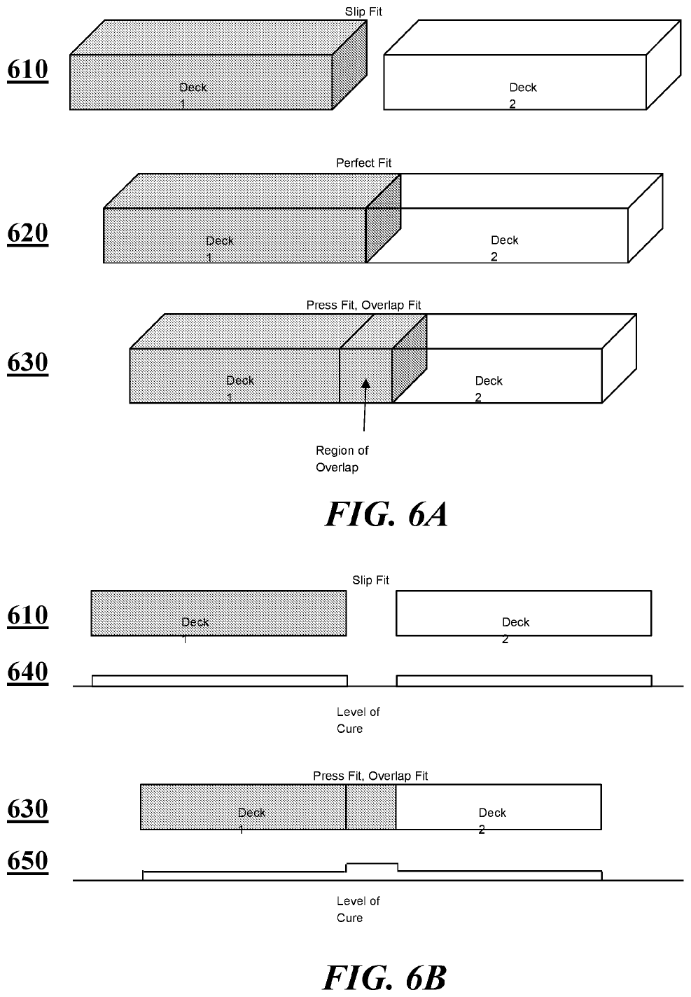

FIGS. 6A-6B show examples of voxel stitching, according to some embodiments. In some cases where a 3D object is larger than a print field of a 3D printer, the 3D object can be divided into a plurality of portions. In some cases, where the 3D printer is capable of printing a volume at once, the 3D object can be divided into a plurality of voxels. The voxels can be printed at different times. The voxels can be printed at a same time. The voxels can be printed as in 610, where the voxels may not be touching (e.g., in a slip fit configuration). The voxels may be held together by non-binding forces (e.g., friction, adsorption, etc.). The structural rigidity of the voxels may be low in a slip fit scheme (e.g., the voxels may move relative to one another with a low amount of force applied). A slip fit configuration may provide looser tolerances than other configurations, which can, in turn be printed on 3D printers with looser tolerances. A perfect fit configuration 620 can comprise printing voxels immediately adjacent to one another (e.g., where the side of one voxel touches the side of another voxel). Such a configuration 620 can use non-binding forces to maintain the positions of the voxels as described elsewhere herein, binding forces (e.g., the overcuring that is present from the production of the voxels that can result in an uncontrolled overlap of the curing of the voxels), or a combination thereof. The binding forces can result in weak adhesion between the voxels, as the level of overcuring may not be well defined and the penetration depth of the overcuring can be shallow. The voxels can be printed as in 630 using an overlapping fit configuration. In the 630 configuration, the voxels can be strongly bound to one another, thereby forming a more continuous structure. The region of overlap can providing region of adequate level of cure that maintains the material properties of the voxels to provide a whole 3D object with such predetermined properties.

FIG. 6B shows an example of the level of cure profiles for a slip fit (610) configuration and overlapping fit (630) configuration, according to some embodiments. The level of cure profiles 640 and 650 can be examples of unitless plots of the level of cure (e.g., level of polymerization) over the voxels. The level of cure plot 640 can show the presence of a gap between the two voxels in a slip fit configuration. The gap may preclude binding forces from being present in the slip fit configuration, as the two voxels may not have any chemical bonds between them. In contrast, the overlapping fit configuration can provide for a single solid object to be formed by the two voxels, as demonstrated by the level of cure profile 650. In this profile, the level of cure of the overlapping region can be double that of the rest of the voxels. This higher level of cure can be due to the overlapping light provided to cure the two voxels. The higher level of cure can result in material properties that deviate from the predetermined material properties of the 3D object. For example, too high of a level of cure can result in brittleness, deviation from a predetermined object size (e.g., overprinting), or the like. To reduce the level of cure of the overlapping region, a different configuration of light can be used.

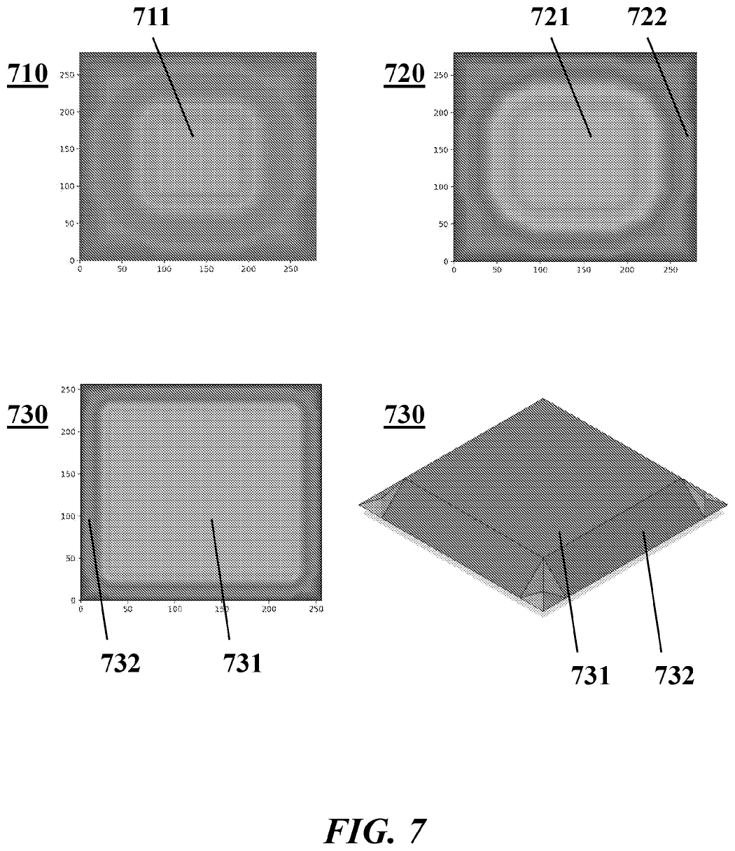

FIG. 7 shows examples of light profiles to reduce overcuring in an overlapping fit configuration, according to some embodiments. The light profiles can be configured with a decreased intensity along the edges of the light profile when compared to the center of the profile. The light profiles can be configured to, when overlapped with one another in a overlapping fit configuration, provide a consistent level of cure across the entirety of both light profiles. For example, the light profiles can be overlapped such that the entire resultant object can have a same predetermined level of cure. A light profile such as that of profile 710 may provide for a smaller high intensity region 711 and a wider gradient of optical intensity 712. Such a wide gradient may enable increased overlap between voxels using profile 710 (e.g., an increased overlap while maintaining a predetermined level of cure). This may, in turn, improve cohesion between the voxels and improve material properties of the completed object. Using a profile such as profile 720 with a larger high intensity region 721 and a smaller gradient of optical intensity 722 may be used for application in which a smaller overlap between voxels has been determined to be beneficial. The gradient of optical intensity may be at least about 0.1, 0.5, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 96, 97, 98, 99, or more percent of the voxel. The gradient of optical intensity may be at most about 99, 98, 97, 96, 95, 90, 85, 80, 75, 70, 65, 60, 55, 50, 45, 40, 35, 30, 25, 20, 15, 14, 13, 12, 11, 10, 9, 8, 7, 6, 5, 4, 3, 2, 1, 0.5, 0.1 or less percent of the voxel. In some cases, a very small gradient can be formed within a voxel, such as that of profile 730 (shown from top-down and isometric views). The small gradient 732 combined with the large high intensity region 731 may enable fast printing of a 3D object due to the minimal overlap between the voxels. The gradient region may comprise a monotonic gradient (e.g., a gradient with only decreasing intensity from the center to the edge of the voxel). The gradient region may comprise a non-monotonic gradient (e.g., a gradient with both increasing and decreasing values over the gradient). The gradient may comprise a linear gradient (e.g., following a linear profile), a geometric gradient (e.g., following a geometric profile), an exponential gradient (e.g., following an exponential gradient), a discontinuous gradient (e.g., comprising one or more discontinuities), or the like, or any combination thereof.

In another aspect, the present disclosure provides a method of troubleshooting a 3D printing process using a 3D printer. The 3D printer can be used to print a first object. The 3D printer may be used to print a second object. The second object can comprise a circle with an equilateral cross disposed therein. The second object can be compared with a computer file for the second object to troubleshoot the 3D printing process.

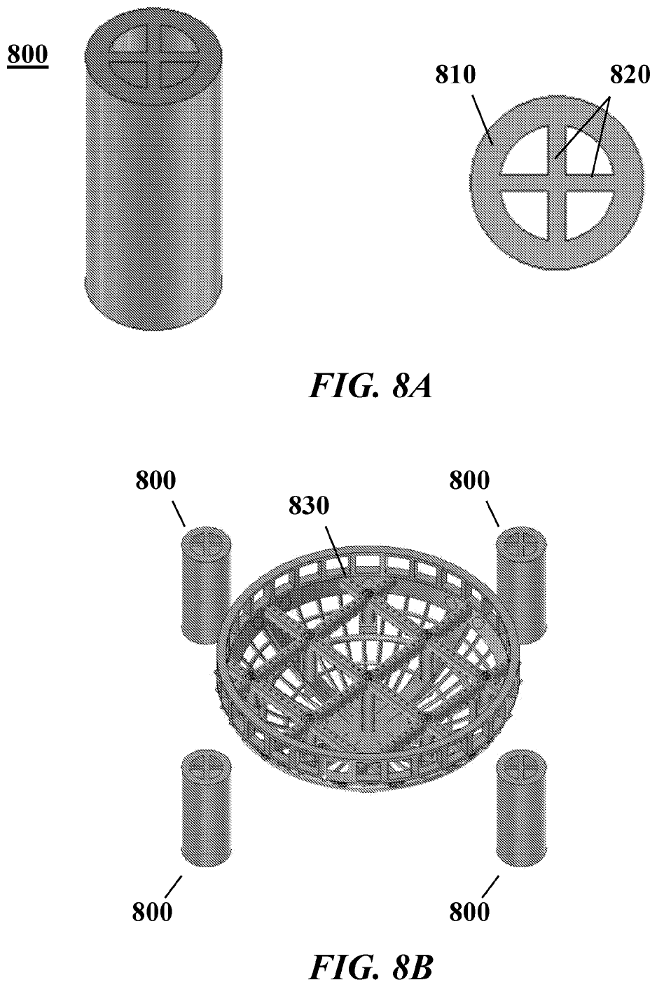

FIG. 8A shows a top and isometric view of an object (e.g., a second object as described elsewhere herein) configured for use as a printing diagnostic, according to some embodiments. FIG. 8B shows an example of a use of an object for a diagnostic during a printing operation, according to some embodiments. The object 800 can comprise a circular portion 810 and one or more cross members 820. The object may comprise at least about 1, 2, 3, 4, 5, 6, 7, 8, 9, 10, or more cross members. The object may comprise at most about 10, 9, 8, 7, 6, 5, 4, 3, 2, or fewer cross members. For example, in FIG. 8A, the object can comprise two cross members. The cross members may be orthogonal to one another. In some cases, the cross members are disposed at an angle from one another. The cross members and the circular portion may be configured such that a misalignment or error of the printing process is apparent in the object. For example, the object comprising the circular portion and the cross bars can be smaller than a 3D object as described elsewhere herein. For example, FIG. 8B shows a plurality of objects 800 surrounding a 3D object 830. In this example, a printing error in the objects 800 may be more readily apparent than a printing error in the center of the 3D object 830. In this way, the objects 800 can provide a more visible and easily tracked indication of print quality. A plurality of objects 800 may be used to track print quality over a wide area. The plurality of objects can be of a same size. The plurality of objects may be of different sizes. For example, a plurality of objects can each be a different size such that printing errors on different scales can be readily observed. In this example, a larger object can be configured to be used to show printing quality of large-scale portions of the 3D object, while a smaller object can be configured to be used to show printing quality of smaller scale portions of the 3D object.

In another aspect, the present disclosure provides a method of troubleshooting a 3D printing process using a 3D printer. The 3D printer may be used to print a first object. The 3D printer may be used to print a second object. The second object may comprise a plurality of cross-hatched lattices. A distance between the lattices may be asymmetrical. The second object may be compared with a computer file for the second object to troubleshoot the 3D printing process.

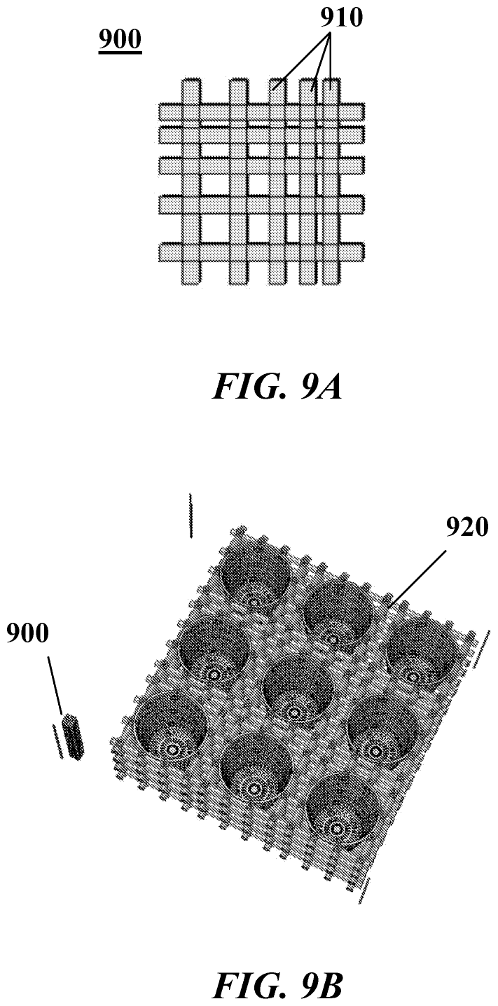

FIG. 9A shows a top view of an object (e.g., a second object as described elsewhere herein) configured for use as a printing diagnostic, according to some embodiments. FIG. 9B shows an example of a use of an object for a diagnostic during a printing operation, according to some embodiments. Object 900 can comprise a plurality of bars 910 configured as a lattice. The lattice may be cross-hatched. The lattice may be asymmetric as show in FIG. 9A. The asymmetry of the lattice may enable detection of printing errors at different scales, for example by using the voids between the cross-hatches as well as the cross-hatches themselves to determine a presence or absence of printing errors. The cross hatches may be of a same size as compared to one another (e.g., as shown in FIG. 9A). The cross hatches may be of different sizes from one another (e.g., along a size gradient from large to small). The object may be printed to a same height as a 3D object. For example, as shown in FIG. 9B, the object 900 can be printed to a same height as the 3D object 920. In this example, not only the in plane print quality can be considered, but the print quality along the height of the object can be used in a quality control assessment of the 3D object.

In some cases, the first object and the second object are printed at a same or substantially same time. For example, the second object can be formed during the formation of the first object. In this way, the second object can indicate issues in the 3D printer that translate into the printing of the first object. For example, if the 3D printer is not aligned properly, the second object can show alignment errors that may not be observable in the first object (e.g., the first object is too large to see interior alignment errors, the first object's feature size is larger than the alignment error, etc.). In this example, without the aid of the second object, the errors in the first object may go unnoticed, and the materials properties of the first object may be detrimentally impacted.

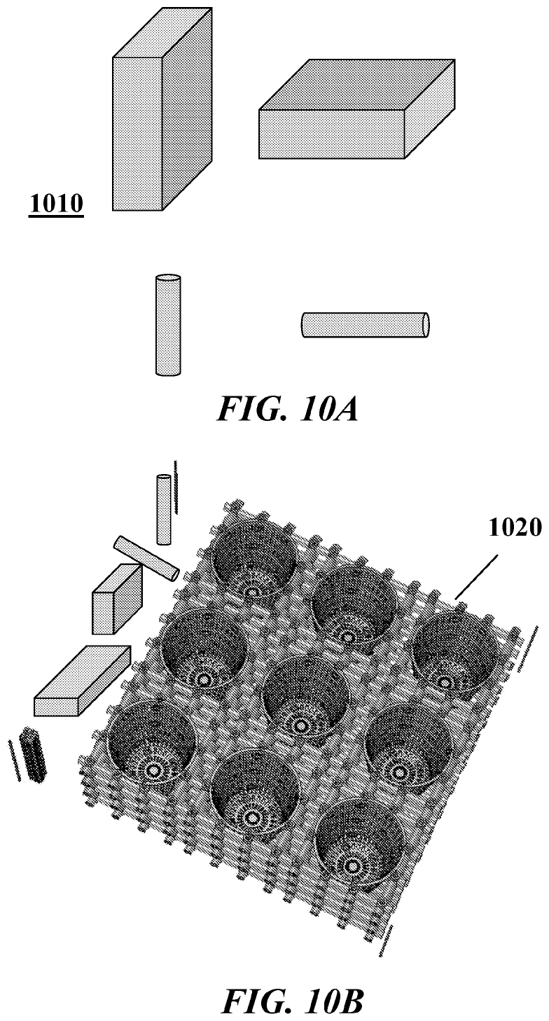

FIG. 10A shows a plurality of objects configured as a diagnostic object (e.g., a second object), according to some embodiments. The plurality of objects 1010 may comprise a plurality of objects as found in the 3D object. For example, the plurality of objects can comprise a plurality of features of the 3D object. In another example, the plurality of objects can comprise a plurality of spokes, walls, tubes, vasculature, or the like, or any combination thereof that is found in the object. Using features of the 3D object as a diagnostic object can provide accurate diagnostic information for the specific features contained within the 3D object. FIG. 10B shows a diagnostic object comprising a plurality of objects 1010 in use next to a 3D object 1020, according to some embodiments. As described elsewhere herein with respect to other diagnostic objects, the diagnostic object can be used to monitor the printing of the 3D object in real time.

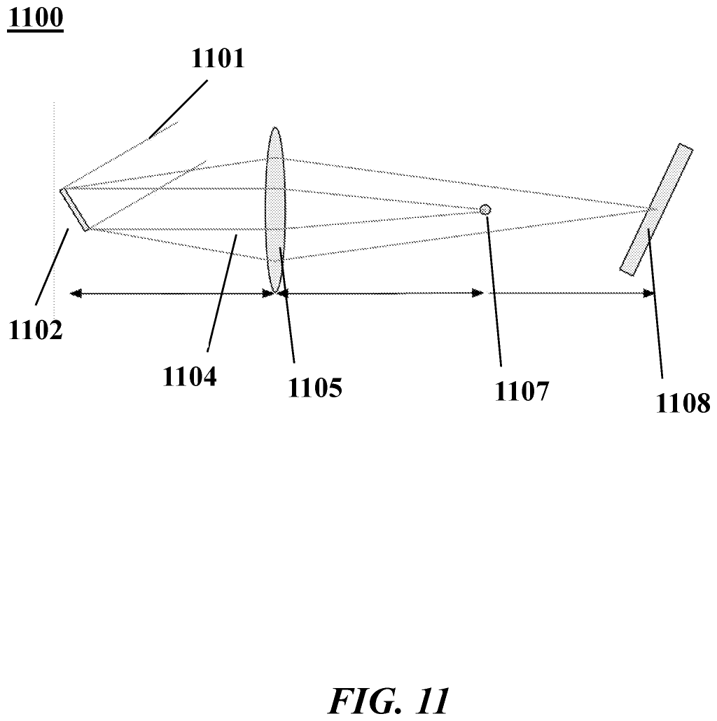

FIG. 11 is an example of a beam blocker, according to some embodiments. The beam blocker 1100 may be configured to reject light that has not been modulated by a spatial light modulator, digital micromirror device, or the like. An incident light beam 1101 may be directed towards an optical element 1102. The optical element 1102 may comprise a spatial light modulator, a digital micromirror device, or the like. The optical element 1102 may be configured to interact with the incident light beam and modulate it to form a modulated light beam 1103. In addition to the modulated light beam, the optical element may also produce an unmodulated light beam 1104. The unmodulated light beam may be a result of a reflection off of a surface of the optical element. The unmodulated light beam may not comprise the light modulations configured to be imparted by the optical element 1102. As such, the unmodulated light beam may result in printing artifacts or other errors if it were to reach a printing medium. In some cases, the unmodulated light beam can comprise about 3%-5% of the total power of the incident light beam.

A second optical element 1105 may be disposed in the light path of the modulated and unmodulated light beams subsequent to the optical element 1102. The second optical element may comprise a lens. The second optical element may be configured to simultaneously focus the modulated light beam onto a third optical element 1106 and the unmodulated light onto a beam block 1107. The third optical element may comprise a mirror, a grating, a filter, or the like. The third optical element may be configured to direct the modulated light beam further into a 3D printer for eventual use in printing a 3D object. The beam block may be configured to remove the unmodulated light from the system. For example, the beam block can be configured to absorb the unmodulated light to remove it from the system. In another example, the beam block can be reflective and configured to reflect the light away from the optical path and out of the system. In some cases, the unmodulated light can be reused by projecting the unmodulated light onto another modulator. The beam block may comprise a solid metal beam block. For example, the beam block can comprise a ball bearing. The beam block may comprise a plurality of fins. For example, the beam block can comprise a plurality of metal fins configured to dissipate heat from the absorption of the unmodulated light. The beam block may comprise a solid non-metal beam block (e.g., a dielectric beam block, a semiconductor beam block, a polymer beam block, etc.).

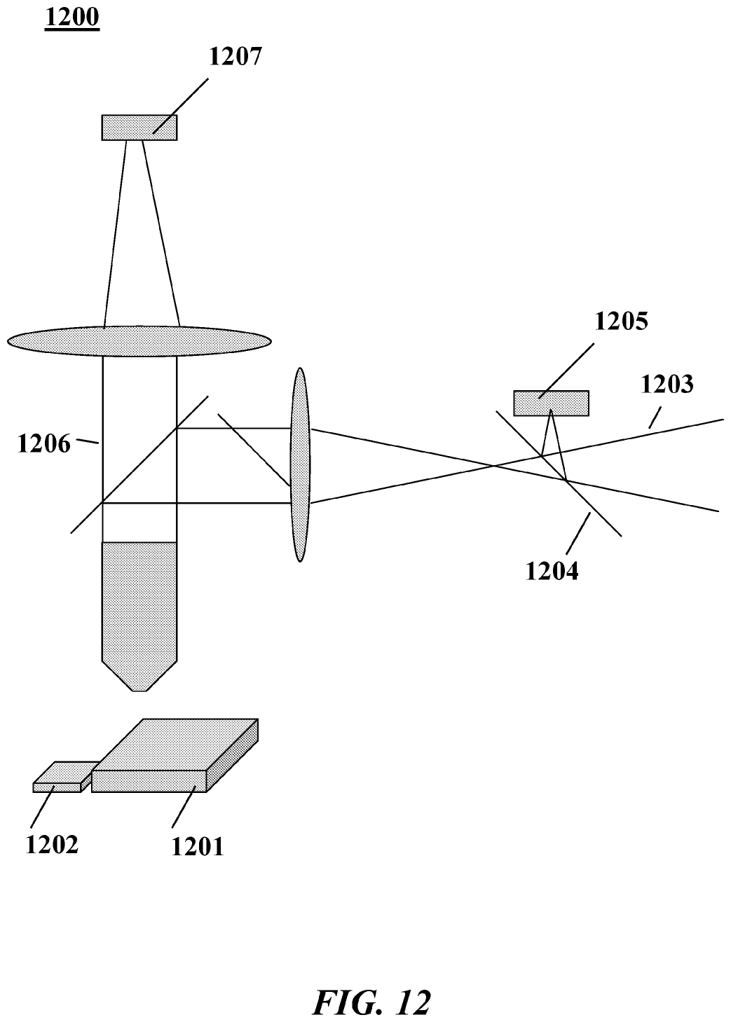

FIG. 12 is an example of a level of cure monitoring configuration 1200 for a 3D printer, according to some embodiments. The level of cure monitoring configuration may be a part of a system as described elsewhere herein (e.g., a 3D printer). The level of cure monitoring system may be configured to monitor a level of cure of a 3D object 1201 or a diagnostic object 1202 (e.g., a second object as described elsewhere herein. The level of cure monitoring system may simultaneously measure the level of cure of a plurality of objects (e.g., both the 3D object and the diagnostic object). The level of cure monitoring system may sequentially measure the level of cure of a plurality of objects (e.g., measure the level of cure of the 3D object and the diagnostic object in an alternating fashion).

The level of cure monitoring system may comprise a light beam 1203. The light beam may be generated by a light source as described elsewhere herein. The light beam may be incident on an optical element 1204. The optical element 1204 may be configured to direct at least a portion of the light beam to a detector 1205. The detector may be a detector as described elsewhere herein. The detector may be configured to monitor the incident light beam. For example, the detector can be configured to monitor the intensity, wavelength, pulse duration, repetition rate, position, or the like, or any combination thereof of the light beam. The detector can provide information about the current and/or past properties of the light beam. Using the information from the detector, the system can make dynamic adjustments to the printing properties based at least in part on the information from the detector. For example, if the detector detects that the power provided by the light beam is decreased over a period of time (e.g., due to fluctuations in the light source), the system can increase dwell time or number of repetitions for the parts of the 3D object that were printed during the period of decreased intensity. In another example, if the detector detects a drift in the position of the light beam, the system can perform an automatic alignment to restore the light beam to the predetermined position. In another example, if the detector detects a drift in the position of the light beam, the system can notify a user to perform a manual adjustment of the alignment.

The level of cure monitoring system may comprise optical elements 1206. The optical elements may be configured to direct at least a portion of the light beam 1203 towards a media bath for printing the 3D object 1201 and/or the diagnostic object 1202. Examples of optical elements that may be included in the level of cure monitoring configuration include, but are not limited to, lenses, mirrors, dichroic filters, beam splitters, objectives, pinholes, apertures, or the like, or any combination thereof. The optical elements 1206 may be configured to direct light from the 3D object and/or the diagnostic object towards a detector 1207. The detector 1207 may be configured as a camera. The detector 1207 may be configured to image at least a portion of a media bath in the system (e.g., at least a portion of a 3D object or diagnostic object). The detector 1207 may be configured to record a plurality of images of the 3D object and/or diagnostic object over time. For example, the detector can be configured to take a video of the printing of the 3D object or the diagnostic object. An image from the detector may be used as an input for a quality control program. For example, a quality control program comprising a machine learning algorithm can use the image from the detector 1207 to quantify the progress of the printing of the 3D object or diagnostic object, and can also use the image to diagnose an issue with the printing. For example, an image of a diagnostic object showing an incomplete cure can be used to alert a user of the problem as well as a starting point for adjusting the printing parameters.

The detector 1207 can be integrated into a fully automated printing process where the 3D object is printed without a user's supervision. For example, a user can provide a file comprising a 3D object to be printed and the system can add a diagnostic object configured to be similar to at least a portion of the elements of the 3D object, print the 3D object and the diagnostic object, monitor the printing of the 3D object and the diagnostic object using one or more detectors, and adjust the printing parameters to optimize the printing of the 3D object and the diagnostic object, all without further input from the user. The fully automated printing process may comprise a plurality of adjustments to the printing parameters over the course of the printing process. For example, the printing parameters can be adjusted for the fluctuations of light beam properties, curing properties, and the like that can occur during a printing process.

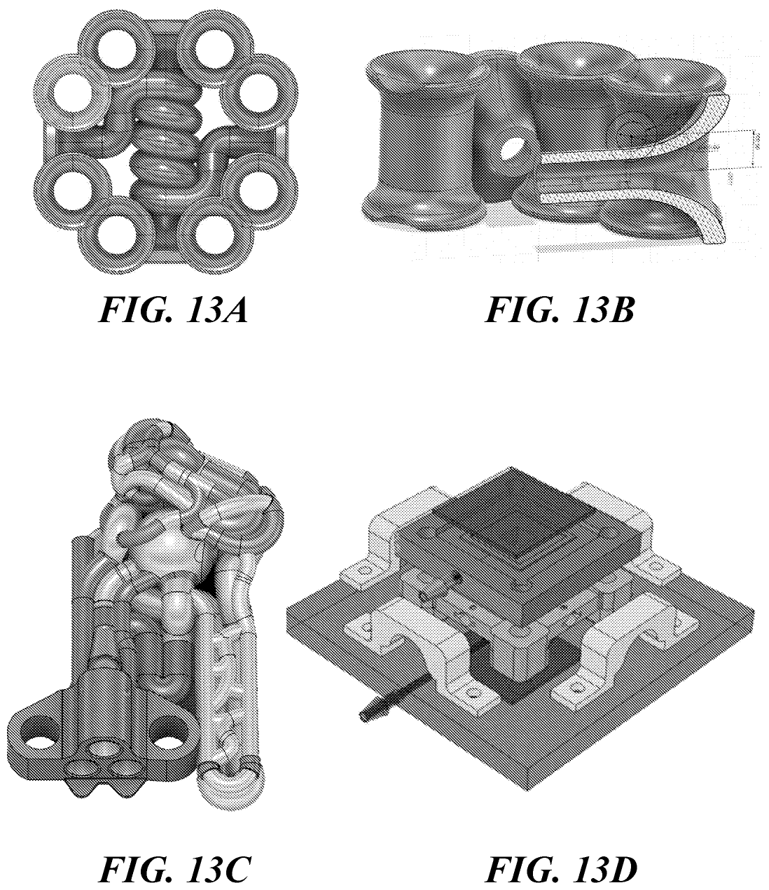

FIGS. 13A-13D are examples of models of a microfluidics platform, according to some embodiments.

### Laser Printing Systems

In an aspect, the present disclosure provides systems for printing a three-dimensional (3D) biological material. The x, y, and z dimensions may be simultaneously accessed by the systems provided herein. A system for printing a 3D biological material may comprise a media chamber configured to contain a medium comprising a plurality of cells comprising cells and one or more polymer precursors. The plurality of cells may comprise cells of at least one type. The plurality of cells may comprise cells of at least two different types. The system may comprise at least one energy source configured to direct at least one energy beam to the media chamber. The system may comprise at least one energy source configured to direct at least one energy beam to the media chamber and/or to the cell-containing chamber. The system may comprise one or more computer processors operatively coupled to the at least one energy source, wherein the one or more computer processors may be individually or collectively programmed to: receive computer instructions for printing the 3D biological material from computer memory; and direct the at least one energy source to direct the at least one energy beam to the medium in the media chamber along at least one energy beam path in accordance with the computer instructions, to subject at least a portion of the polymer precursors to form at least a portion of the 3D biological material.

In another aspect, the present disclosure provides an additional system for printing a 3D biological material, comprising a media chamber configured to contain a medium comprising a plurality of cells and a plurality of polymer precursors. The system may comprise at least one energy source configured to direct at least one energy beam to the media chamber. In addition, the system may comprise one or more computer processors that may be operatively coupled to the at least one energy source. The one or more computer processors may be individually or collectively programmed to: (i) receive computer instructions for printing the 3D biological material from computer memory; (ii) direct the at least one energy source to direct the at least one energy beam to the medium in the media chamber along at least one energy beam path in accordance with the computer instructions, to subject at least a portion of the polymer precursors to form at least a portion of the 3D biological material; and (iii) direct the at least one energy source to direct the at least one energy beam to a second medium in the media chamber along at least one energy beam path in accordance with the computer instructions, to subject at least a portion of the second medium in the media chamber to form at least a second portion of the 3D biological material, wherein the second medium comprises a second plurality of cells and a second polymeric precursor, wherein the second plurality of cells is of a different type than the first plurality of cells. The laser printing system may comprise a laser printing system as described in PCT publication number PCT/US2020/052897, the disclosure of which is incorporated by reference in its entirety.

The one or more computer processors are individually or collectively programmed to generate a point-cloud representation or lines-based representation of the 3D biological material in computer memory, and use the point-cloud representation or lines-based representation to generate the computer instructions for printing the 3D biological material in computer memory. The one or more computer processors may be individually or collectively programmed to direct the at least one energy source to direct the at least one energy beam along one or more additional energy beam paths to form at least another portion of the 3D biological material.

The system may comprise one or more computer processors operatively coupled to at least one energy source and/or to at least one light patterning element. The point-cloud representation or the lines-based representation of the computer model may be a holographic point-cloud representation or a holographic lines-based representation. The one or more computer processors may be individually or collectively programmed to use the light patterning element to re-project the holographic image as illuminated by the at least one energy source.

In some cases, one or more computer processors may be individually or collectively programmed to convert the point-cloud representation or lines-based representation into an image. The one or more computer processors may be individually or collectively programmed to project the image in a holographic manner. The one or more computer processors may be individually or collectively programmed to project the image as a hologram. The one or more computer processors may be individually or collectively programmed to project the image as partial hologram. In some cases, one or more computer processors may be individually or collectively programmed to convert the point-cloud representation or lines-based representation of a complete image set into a series of holographic images via an algorithmic transformation.

This transformed image set may then be projected in sequence by a light patterning element, such as a spatial light modulator (SLM) or digital mirror device (DMD), through the system, recreating the projected image within the printing chamber with the projected light that is distributed in 2D and or 3D simultaneously. An expanded or widened laser beam may be projected onto the SLMs and/or DMDs, which serve as projection systems for the holographic image.

In some cases, in addition or in alternative to the SLMs and/or DMDs, the system, when used, e.g., for holographic laser printing, comprises a liquid lens. For example, the liquid lens may be used in place of the SLM or the DMD. In some cases, unlike glass lenses, the liquid lens may comprise single optical elements comprising an optical liquid material that may be able to change in shape. While focal length of a glass lens may be dependent on the material the glass lens is made from and the radius of curvature of the glass lens, focal length of the liquid lens may be alterable by changing the radius of curvature. The radius of curvature for the liquid lens may be electronically controllable and may be rapidly changed (e.g., on the order of milliseconds). In some cases, the liquid lens may implement electrowetting, shape-changing polymers, or acusto-optical tuning techniques to control the radius of curvature and refractive index of the liquid lens.

In some cases, one or more computer processors may be individually or collectively programmed to project the image in a holographic manner. In some cases, one or more computer processors may be individually or collectively programmed to project the images all at once or played in series as a video to form a larger 3D structure in a holographic manner.

Holography is a technique that projects a multi-dimensional (e.g. 2D and/or 3D) holographic image or a hologram. When a laser that can photo-polymerize a medium is projected as a hologram, the laser may photopolymerize, solidify, cross-link, bond, harden, and/or change a physical property of the medium along the projected laser light path; thus, the laser may allow for the printing of 3D structures. Holography may require a light source, such as a laser light or coherent light source, to create the holographic image. The holographic image may be constant over time or varied with time (e.g., a holographic video). Furthermore, holography may require a shutter to open or move the laser light path, a beam splitter to split the laser light into separate paths, mirrors to direct the laser light paths, a diverging lens to expand the beam, and additional patterning or light directing elements.

A holographic image of an object may be created by expanding the laser beam with a diverging lens and directing the expanded laser beam onto the hologram and/or onto at least one pattern forming element, such as, for example a spatial light modulator or SLM. The pattern forming element may encode a pattern comprising the holographic image into a laser beam path. The pattern forming element may encode a pattern comprising a partial hologram into a laser beam path. Next, the pattern may be directed towards and focused in the medium chamber containing the printing materials (i.e., the medium comprising the plurality of cells and polymeric precursors), where it may excite a light-reactive photoinitiator found in the printing materials (i.e., in the medium). Next, the excitation of the light-reactive photoinitiator may lead to the photopolymerization of the polymeric-based printing materials and forms a structure in the desired pattern (i.e., holographic image). In some cases, one or more computer processors may be individually or collectively programmed to project the holographic image by directing an energy source along distinct energy beam paths.

In some cases, at least one energy source may be a plurality of energy sources. The plurality of energy sources may direct a plurality of the at least one energy beam. The energy source may be a laser. In some examples, the laser may be a fiber laser. For example, a fiber laser may be a laser with an active gain medium that includes an optical fiber doped with rare-earth elements, such as, for example, erbium, ytterbium, neodymium, dysprosium, praseodymium, thulium and/or holmium. The energy source may be a short-pulsed laser. The energy source may be a femto-second pulsed laser. The femtosecond pulsed laser may have a pulse width less than or equal to about 500 femtoseconds (fs), 250, 240, 230, 220, 210, 200, 150, 100, 50 fs, 40 fs, 30 fs, 20 fs, 10 fs, 9 fs, 8 fs, 7 fs, 6 fs, 5 fs, 4 fs, 3 fs, 2 fs, 1 fs, or less. The femtosecond pulsed laser may be, for example, a titanium:sapphire (Ti:Sa) laser. The at least one energy source may be derived from a coherent light source.

The coherent light source may provide light with a wavelength from about 300 nanometers (nm) to about 5 millimeters (mm). The coherent light source may comprise a wavelength from about 350 nm to about 1800 nm, or about 1800 nm to about 5 mm. The coherent light source may provide light with a wavelength of at least about 300 nm, 400 nm, 500 nm, 600 nm, 700 nm, 800 nm, 900 nm, 1 mm, 1.1 mm, 1.2, mm, 1.3 mm, 1.4 mm, 1.5 mm, 1.6 mm, 1.7 mm, 1.8 mm, 1.9 mm, 2 mm, 3 mm, 4 mm, 5 mm, or greater.

The computer processors may be individually or collectively programmed to direct the at least one energy source to direct the at least one energy beam along one or more additional energy beam paths to form at least another portion of the 3D biological material. The one or more additional energy beam paths may be along an x axis, an x and y plane, or the x, y, and z planes. The one or more additional energy beam paths may be along an x axis. The one or more additional energy beam paths may be along a y axis. The one or more additional energy beam paths may be along a z axis. The energy beam path may converge with one or more other beams on the same axis. The one or more additional energy beam paths may be in the x and y plane. The one or more additional energy beam paths may be in the x and z plane. The one or more additional energy beam paths may be in the y and z plane. The one or more additional energy beam paths may be in the x, y, and z planes.

The system may further comprise at least one objective lens for directing the at least one energy beam to the medium in the media chamber. In some instances, at least one objective lens may comprise a water-immersion objective lens. In some instances, at least one objective lens may comprise a water-immersion objective lens. In some instances, at least one objective lens may comprise a water dipping objective lens. In some instances, at least one objective lens may comprise an oil immersion objective lens. In some instances, at least one objective lens may comprise an achromatic objective lens, a semi-apochromatic objective lens, a plans objective lens, an immersion objective lens, a Huygens objective lens, a Ramsden objective lens, a periplan objective lens, a compensation objective lens, a wide-field objective lens, a super-field objective lens, a condenser objective lens, or any combination thereof. Non-limiting examples of a condenser objective lens may include an Abbe condenser, an achromatic condenser, and a universal condenser.

The one or more computer processors may be individually or collectively programmed to receive images of the edges of the 3D biological material. The one or more computer processors may be individually or collectively programmed to receive images of the exterior surfaces of the 3D biological material. The one or more computer processors may be individually or collectively programmed to receive images of the interior surfaces of the 3D biological material. The one or more computer processors may be individually or collectively programmed to receive images of the interior of the 3D biological material.

The one or more computer processors may be individually or collectively programmed to direct linking of the 3D biological material with other tissue, which linking may be in accordance with the computer instructions. The one or more computer processors may be individually or collectively programmed to directly link, merge, bond, or weld 3D printed material with already printed structures, where linking is in accordance with the computer model. In some cases, linking of the 3D biological material with other tissue may involve chemical cross-linking, mechanical linking, and/or cohesively coupling.

In another aspect, the system may comprise a media chamber configured to contain a medium comprising a plurality of cells and a plurality of polymer precursors. The system may comprise at least one energy source configured to direct at least one energy beam to the media chamber. The system may comprise one or more computer processors operatively coupled to at least one energy source, wherein the one or more computer processors are individually or collectively programmed to: receive a computer model of the 3D biological material in computer memory; generate a point-cloud representation or lines-based representation of the computer model of the 3D biological material in computer memory; direct the at least one energy source to direct the at least one energy beam to the medium in the media chamber along at least one energy beam path in accordance with the computer model of the 3D biological material, to subject at least a portion of the polymer precursors to form at least a portion of the 3D biological material; and direct the at least one energy source to direct the at least one energy beam to a second medium in the media chamber along at least one energy beam path in accordance with the computer model of the 3D biological material, to subject at least a portion of the second medium in the media chamber to form at least a second portion of the 3D biological material, wherein the second medium comprises a second plurality of cells and a second polymeric precursor, wherein the second plurality of cells is of a different type than the first plurality of cells.

In laser printing of cellular structures, rapid three-dimensional structure generation using minimally toxic laser excitation is critical for maintaining cell viability and in the case of functional tissue printing, necessary for large-format, high resolution, multicellular tissue generation. Other methods of two-photon printing may rely upon raster-scanning of two-photon excitation in a two-dimensional plane (x, y) (e.g., selective laser sintering), while moving the microscope or stage in the z direction to create a three-dimensional structure. This technique may be prohibitively slow for large format multicellular tissue printing such that cell viability may be unlikely to be maintained during printing of complex structures. Certain hydrogels with high rates of polymerization may also be utilized for two-dimensional projection of tissue sheets that are timed such that one slice of a structure is projected with each step in in an x, y, or z plane. Additionally, mixed plane angles representing a sheet or comprising an orthogonal slice may also be utilized. In the case of rapidly polymerizing hydrogels, these projections may work in time-scales that are compatible with tissue printing whereas laser sintering or raster scanning (e.g. layer-by-layer deposition) may be prohibitively slow for building a complex structure.

The laser printing system may comprise a half wave plate. The half wave plate may comprise crystalline quartz (SiO₂), calcite (CaCO₃), magnesium fluoride (MgF₂), sapphire (Al₂O₃), mica, or a birefringent polymer. The laser printing system may comprise an energy source. The energy source (e.g., laser) may provide energy (e.g., laser beam) having a wavelength from about 300 nm to 5 mm, 600 nm to 1500 nm, 350 nm to 1800 nm, or 1800 nm to 5 mm. The energy source (e.g., laser) may provide energy (e.g., laser beam) having a wavelength of at least about 300 nm, 400 nm, 500 nm, 600 nm, 700 nm, 800 nm, 900 nm, 1 mm, 1.1 mm, 1.2, mm, 1.3 mm, 1.4 mm, 1.5 mm, 1.6 mm, 1.7 mm, 1.8 mm, 1.9 mm, 2 mm, 3 mm, 4 mm, 5 mm, or greater. The energy source may be an energy beam, a heat source, or a light source. The energy source may be a laser, such as a fiber laser, a short-pulsed laser, or a femto-second pulsed laser. The energy source may be a heat source, such as a thermal plate, a lamp, an oven, a heated water bath, a cell culture incubator, a heat chamber, a furnace, a drying oven, or any combination thereof. The energy source may be a light source, such as white light, infrared light, ultraviolet (UV) light, near infrared (NIR) light, visible light, a light emitting diode (LED), or any combination thereof. The energy source may be a sound energy source, such as an ultrasound probe, a sonicator, an ultrasound bath, or any combination thereof. The energy source may be an electromagnetic radiation source, such as a microwave source, or any combination thereof.

### Methods of Printing Organs and Organoids

The present disclosure provides methods and systems for producing one or more immunological proteins. In an aspect, a method for producing one or more immunological proteins comprises providing a media chamber comprising a medium comprising: (i) a plurality of cells and (ii) one or more polymer precursors. Next, at least one energy beam may be directed to the medium in the media chamber along at least one energy beam path that is patterned into a three-dimensional (3D) projection in accordance with computer instructions for printing a 3D lymphoid organoid in computer memory. This may form at least a portion of the 3D lymphoid organoid comprising: (i) at least a subset of the plurality of cells, and (ii) a polymer formed from the one or more polymer precursors. Next, a method for producing one or more immunological proteins may comprise subjecting the at least one portion of the 3D lymphoid organoid to conditions sufficient to stimulate production of the one or more immunological proteins.

In another aspect, a method for producing one or more immunological proteins, comprises (i) printing a three-dimensional (3D) lymphoid organoid comprising a matrix containing a plurality of cells, and (ii) treating the 3D lymphoid organoid to produce the one or more immunological proteins.

In another aspect, a method for producing one or more immunological proteins, comprises: providing a media chamber comprising a first medium. The first medium may comprise a first plurality of cells and a first polymeric precursor. Next, at least one energy beam may be directed to the first medium in the media chamber along at least one energy beam path in accordance with computer instructions for printing a three-dimensional (3D) lymphoid organoid in computer memory, to subject at least a portion of the first medium in the media chamber to form a first portion of the 3D lymphoid organoid. Next, the method may provide a second medium in the media chamber. The second medium may comprise a second plurality of cells and a second polymeric precursor. The second plurality of cells may be of a different type than the first plurality of cells. Next, the method may comprise directing at least one energy beam to the second medium in the media chamber along at least one energy beam path in accordance with the computer instructions, to subject at least a portion of the second medium in the media chamber to form a second portion of the 3D lymphoid organoid. Next, the method may comprise subjecting the first and second portions of the 3D lymphoid organoid to conditions sufficient to stimulate production of the one or more immunological proteins.

In another aspect, a method of producing one or more immunological proteins comprises (i) printing a three-dimensional (3D) lymphoid organoid comprising a matrix containing a first plurality of cells and a second plurality of cells, and (ii) treating the 3D lymphoid organoid to produce the one or more immunological proteins.

Another aspect of the present disclosure provides a system for producing one or more immunological proteins, comprising a media chamber configured to contain a medium comprising a plurality of cells and one or more polymer precursors. The system may comprise at least one energy source configured to direct at least one energy beam to the media chamber. The system may comprise one or more computer processors operatively coupled to the at least one energy source. The one or more computer processors may be individually or collectively programmed to receive computer instructions for printing a three-dimensional (3D) lymphoid organoid from computer memory. The one or more computer processors may be individually or collectively programmed to direct the at least one energy source to direct the at least one energy beam to the medium in the media chamber along at least one energy beam path in accordance with the computer instructions, to subject at least a portion of the polymer precursors to form at least a portion of the 3D lymphoid organoid. The one or more computer processors may be individually or collectively programmed to subject the at least portion of the 3D lymphoid organoid to conditions sufficient to stimulate production of the one or more immunological proteins. The one or more computer processors may be individually or collectively further programmed to extract one or more immunological proteins from the at least portion of the 3D lymphoid organoid.

Another aspect of the present disclosure provides a method of producing a population of human immunological proteins, comprising: using a multi-photon laser bio-printing system to bio-print a three-dimensional lymphoid organoid. Next, the method may comprise exposing the three-dimensional lymphoid organoid to an antigen in order to stimulate production of the population of human immunological proteins. Next, the method may comprise extracting the population of human immunological proteins from the three-dimensional lymphoid organoid.

The conditions sufficient to stimulate production of the one or more immunological proteins may comprise exposing at least a portion of the 3D lymphoid organoid to an antigen in order to stimulate production of the one or more immunological proteins. The antigen may be selected from the list consisting of whole peptides, partial peptides, glycopeptides, whole proteins or protein subunits, carbohydrates, nucleic acids, live virus, heat-killed virus, viral particles, membrane bound or stabilized proteins, phage displayed antigens and whole cells. The antigen may be an exogenous antigen, an endogenous antigen, an autoantigen, a neoantigen, or a combination thereof. A neoantigen is defined herein as an antigen that is absent from a normal human genome. The neoantigen may be a tumor antigen, a viral antigen, an engineered antigen, or a synthetic antigen.

Methods of the present disclosure may further comprise extracting one or more immunological proteins from the at least portion of the 3D lymphoid organoid. The one or more immunological proteins may be human immunological proteins. The immunological proteins may be selected from the list consisting of antibodies, T-cell receptors, and cancer immunotherapeutics. The antibodies may be immunoglobulin G (IgG) antibodies. The IgG antibodies may be human IgG antibodies. The immunological proteins may be IgM, IgA, IgE, IgD antibodies or a combination thereof. The immunological proteins may be antibody fragments, antibody domains, immunoglobulin heavy chains, immunoglobulin light chains, or a combination thereof. The antibody fragments may be antigen-binding fragments (Fab), single chain variable fragments (scFv), or a combination thereof. The immunological proteins may be multivalent recombinant antibodies. The multivalent recombinant antibodies may be diabodies (i.e., small recombinant bispecific antibodies), minibodies (i.e., engineered antibody fragments), triabodies, tetrabodies, or a combination thereof. The immunological proteins may be engineered immunological proteins, synthetic immunological proteins, or a combination thereof. The synthetic immunological proteins may be nucleic acid aptamers, non-immunoglobulin protein scaffolds, non-immunoglobulin peptide aptamers, affimer proteins, or a combination thereof.

The plurality of cells may be from a subject. The plurality of cells may be autologous. The plurality of cells may be allogeneic. The plurality of cells may be selected from the list consisting of stromal endothelial cells, endothelial cells, follicular reticular cells or precursors thereof, naïve B cells or other immature B cells, memory B cells, plasma B cells, helper T cells and subsets of the same, effector T cells and subsets of the same CD+8 T cells, CD4+ T cells, regulatory T cells, natural killer T cells, naïve T cells or other immature T cells, dendritic cells and subsets of the same, follicular dendritic cells, Langerhans dendritic cells, dermally-derived dendritic cells, dendritic cell precursors, monocyte-derived dendritic cells, monocytes and subsets of the same macrophages and subsets of the same, leukocytes and subsets of the same. The B cells may be selected from the list consisting of naïve B cells, mature B cells, plasma B cells, B1 B cells, and B2 B cells. The T cells may be selected from the list consisting of CD8+ and CD4+.

The 3D lymphoid organoid may be selected from the list consisting of a B cell germinal center, a thymic-like development niches, a lymph node, an islet of Langerhans, a hair follicle, a tumor, tumor spheroid, a neural bundle or support cells, a nephron, a liver organoid, an intestinal crypt, a primary lymphoid organ, and a secondary lymphoid organ. The shape of the 3D lymphoid organoid may be selected from the list consisting of spherical, oval, ovate, ovoid, square, rectangular, cuboid, any polygonal shape, free-form, and tear-drop shape. The shape of the 3D lymphoid organoid may be a tear-drop shape.

The polymer of the at least of the portion of 3D lymphoid organoid may form a network. The polymer may be collagen, hyaluronic acid and other glycosaminoglycans, poly-dl-lactic-co-glycolic acid (PLGA), poly-l-lactic acid (PLLA), polyglycolic acid (PGA), alginate, gelatin, agar, or a combination thereof. The polymer may comprise an extracellular matrix component. Non-limiting examples of extracellular matrix components used to create 3D lymphoid organoids may include proteoglycans such as heparan sulfate, chondroitin sulfate, and keratan sulfate, non-proteoglycan polysaccharide such as hyaluronic acid, collagen, and elastin, fibronectin, laminin, nidogen, or any combination thereof. These extracellular matrix components may be functionalized with acrylate, diacrylate, methacrylate, cinnamoyl, coumarin, thymine, or other side-group or chemically reactive moiety to facilitate cross-linking induced directly by multi-photon excitation or by multi-photon excitation of one or more chemical doping agents. In some cases, photopolymerizable macromers and/or photopolymerizable monomers may be used in conjunction with the extracellular matrix components to create cell-containing structures. Non-limiting examples of photopolymerizable macromers may include polyethylene glycol (PEG) acrylate derivatives, PEG methacrylate derivatives, and polyvinyl alcohol (PVA) derivatives. In some instances, collagen used to create cell containing structure may be fibrillar collagen such as type I, II, III, V, and XI collagen, facit collagen such as type IX, XII, and XIV collagen, short chain collagen such as type VIII and X collagen, basement membrane collagen such as type IV collagen, type VI collagen, type VII collagen, type XIII collagen, or any combination thereof.

The polymer of the at least of the portion of 3D lymphoid organoid may contain other polymerizable monomers that are synthesized and not native to mammalian tissues, comprising a hybrid of biologic and synthetic materials. An example mixture may comprise about 0.4% w/v collagen methacrylate plus the addition of about 50% w/v polyethylene glycol diacrylate (PEGDA). Photoinitiators to induce polymerization may be reactive in the ultraviolet (UV), infrared (IR), or visible light range. Examples of two such photo initiators are Eosin Y (EY) and triethanolamine (TEA), that when combined may polymerize in response to exposure to visible light (e.g., wavelengths of about 390 to 700 nanometers). Non-limiting examples of photoinitiators may include azobisisobutyronitrile (AIBN), benzoin derivatives, benziketals, hydroxyalkylphenones, acetophenone derivatives, trimethylolpropane triacrylate (TPT), acryloyl chloride, benzoyl peroxide, camphorquinone, benzophenone, thioxanthones, and 2-hydroxy-1-[4-(hydroxyethoxy)phenyl]-2-methyl-1-propanone. Hydroxyalkylphenones may include 4-(2-hydroxyethylethoxy)-phenyl-(2-hydroxy-2-methyl propyl) ketone (Irgacure® 295), 1-hidroxycyclohexyl-1-phenyl ketone (Irgacure® 184) and 2,2-dimethoxy-2-phenylacetophenone (Irgacure® 651). Acetophenone derivatives may include 2,2-dimethoxy-2-phenylacetophenone (DMPA). Thioxanthones may include isopropyl thioxanthone.

The network, formed by the polymer, may be reticular, amorphous, or a net. The net may be an organized net. The organized net may comprise a repeated pattern. The network may be a structured network. The network may be an unstructured network. The network may be a hybrid grid wherein it comprises a mixture of structured and unstructured portions. The network may be a two-dimensional network. The network may be a three-dimensional network. The three-dimensional network may be a tetrahedron network, a pyramidal network, a hexahedron network, a polyhedron network, or a combination thereof. The network, formed by the polymer, may be a mesh. The mesh may be a triangular mesh, an octagonal mesh, a hexagonal mesh, a rectangular mesh, a square mesh, a diamond mesh, a circular mesh, or a combination thereof. The mesh may have varying sizes of each cell per unit area. The amorphous network may be designed to facilitate cellular interactions. The cellular interactions may be B cell to T cell conjugate formation, B cell to B cell interactions, B cell to macrophage, T cell to dendritic cell interactions, stromal cell interactions with T cells, stromal cell interactions with B cells, or stromal cell interactions with dendritic cells. The amorphous network may be designed to facilitate movement between or within cellular niches.

In an aspect, the present disclosure provides a method of printing an organ and/or an organoid. The method may comprise polymerization of a photopolymerizable material by a laser light source. The organ and/or the organoid may be two-dimensional or three-dimensional. The organ and/or the organoid may be a lymph node. The organoid may be an islet of Langerhans. The organoid may be a hair follicle. The organ and/or the organoid may be a tumor and/or a tumor spheroid. The organoid may be a neural bundle and support cells such as, but not limited to Schwann cells and glial cells including satellite cells, olfactory ensheathing cells, enteric glia, oligodendroglia, astroglia, and/or microglia. The organoid may be a nephron. The organoid may be a liver organoid. The organoid may be an intestinal crypt. The organ and/or the organoid may be a primary lymphoid organ, a secondary lymphoid organ such as a spleen, a liver, a pancreas, a gallbladder, an appendix, a brain, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and/or a blood vessel.

Organization of an organ or organoid through the printing process, disclosed herein, may require or be implemented by the sequential deposition of at least about 1, 10, 50, 100, 200, 300, 500, 600, 700, 800, 900, 1000, 10000, 100000, 1000000 or more layers of cells. Organization of a lymphoid organ through the printing process may require or be implemented by the sequential deposition of between 1 and 100 layers of cells. The size of a layer of cells may be tissue dependent. The size of a layer of cells may comprise a larger three-dimensional structure that may be one layer of cells or may comprise multiple layers of cells. The layer of cells may comprise about at least 10, 10², 10³, 10⁴, 10⁵, 10⁶, 10⁷, 10⁸, 10⁹, 10¹⁰, or more cells. Where precise placement of each cell type relative to the other is desired, cells should be printed in sequential steps with a wash step in between to remove the previously used media. Alternately, two or more cell types of different sizes may be printed simultaneously using two photopolymerizable materials of different polymerization wavelength and pore size, such that the larger cell type may become encapsulated in the pore of larger size and the smaller cell type may become encapsulated in the pore of smaller size. Cells are encapsulated in pores in accordance with the size of their nucleus, as the cytoskeleton is able to remodel based on the available space.

The laser light source may use high-energy green, blue, white, or lower frequencies of ultraviolet light to induce polymerization of the photopolymerizable material, or a high-resolution multi-photon light source of any wavelength may be used. The high-resolution, nontoxic multi-photon projection technology is uniquely suited to print detailed germinal centers that allow for the development of light and dark zones that recapitulate natural B cell affinity maturation. This method may be used in combination with microfluidic manipulation of vasculature, whether lymphatic or circulatory, to create functional collagen-based organs and/or organoids, such as lymph node organoids. Nontoxic wavelengths of visible and ultraviolet light may alternatively be used to print cell-containing structures or biogels to be seeded with cells.’

The cells may be introduced to the media chamber and/or the 3D object subsequent to the printing of the 3D object. The cells may be present in the media chamber prior to the printing of the 3D object.

Native architecture may be obtained from imaging data and rendered into two- or three-dimensional images with defined edges and/or grey areas, which are edges that are not precisely defined, but fall somewhere within a designated range, for projection into a polymerizable hydrogel. Such imaging data may provide sufficient detail to enable precise re-creation of multicellular niches that support cell-cell interactions during an immune response. Multicellular niches are developed in the immune system for single B- or T-cell selection based on receptor recognition of a foreign pathogen or material. High reactivity of a receptor or high affinity recognition during an immune response leads to selection for that B or T cell and further cell division and expansion of the numbers of cells that express the highly reactive receptor. Competition for survival signals transmitted by the receptor that is highly reactive in these multicellular niches leads to positive selection of the most reactive B or T cell. Native lymph node architecture can support the development of this selection process which is dependent upon a sequence of specific cell-cell interactions that support selection and proliferation of the highly reactive cells. Therefore, three-dimensional native architecture that allows for cell-cell interactions and independent cell movement is a critical component of the B-cell and T-cell clonal selection process. As such, this architecture is an important component of the printed lymph node and one that is afforded especially by the use of multi-photon lasers in the printing process, though it may be possible to achieve function without printing in this level of resolution achieved with projection of wave-front shaped multi-photon laser light.

Cell-cell interactions that may occur within a multicellular niche include, but are not limited to: B cell-T cell conjugate formation, B cell B cell interactions, B cell macrophage, T cell-dendritic cell interactions), and stromal cell interactions with T, B and Dendritic cells. Interactions are not distinctly paired interactions and clusters or clumps of cells of various types often form during an immune reaction, especially in an established cellular niche or tissue like structure.

T cells, as used here, may refer to any form of a T cell including but not limited to CD8+ or CD4+ T cells. B cells may refer to B cells in any developmental phase including but not limited to naive B cells, mature B cells, plasma B cells, B1 B cells, or B2 B cells.

Multiple organoid units may be printed within a single structure to produce larger organs, up to and including a fully sized organ. Multiple lymphoid units may be printed within a single structure to produce larger immune organs, up to and including a fully sized lymph node or thymus. The limiting factor for size is vascularization, which is essential for tissues larger than 200 micron in width due to the diffusion limits of most gases and nutrients. The completed lymphoid organ or organoid may be between 50 and 200 microns thick without vascularization. If vascularized, the tissue may be 50 microns to 10 cm thick, may be of any shape or size, and may contain both circulatory and lymphatic vasculature. Vasculature may include valves and/or sphincters. In some embodiments, vasculature may be achieved by printing endothelial cells or precursors thereof within a net 500 intended to closely resemble native microvasculature, the structure of which is obtained from high-resolution imaging data. Capillary beds may branch from larger arterioles and arteries and branch into venules and veins in accordance with the relevant anatomy.

The medium may be physically polymerized in order to form a biogel. The medium may be polymerized by a heat source in order to form a biogel. The medium may be chemically polymerized in order to form a biogel; for example, by use of a cross-linker. Non-limiting examples of cross-linkers include 1-ethyl-3-(3-dimethylaminopropyl)-carbodiimide (EDC), glutaraldehyde, and 1-ethyl-3-3-dimethyl aminopropyl carbodiimide (EDAC). The medium may comprise a photoinitiator, a cross-linker, collagen, hyaluronic acid and other glycosaminoglycans, poly-dl-lactic-co-glycolic acid (PLGA), poly-l-lactic acid (PLLA), polyglycolic acid (PGA), alginate, gelatin, agar, or any combination thereof. The biogel may comprise a photoinitiator, a cross-linker, collagen, hyaluronic acid and other glycosaminoglycans, poly-dl-lactic-co-glycolic acid (PLGA), poly-l-lactic acid (PLLA), polyglycolic acid (PGA), alginate, gelatin, agar, or any combination thereof. The polymer precursor may be collagen, hyaluronic acid and other glycosaminoglycans, poly-dl-lactic-co-glycolic acid (PLGA), poly-l-lactic acid (PLLA), polyglycolic acid (PGA), alginate, gelatin, agar, or any combination thereof.

The biogel may be a hydrogel. The biogel may be a biocompatible hydrogel. The biogel may be a polymeric hydrogel. The biogel may be a hydrogel bead. The biogel may be a hydrogel nanoparticle. The biogel may be a hydrogel droplet. The biogel may be a hydrogel microdroplet.

### Methods of Printing Cell-Containing Structures

The present disclosure provides methods and systems of printing and using a three-dimensional cell-containing matrix. In an aspect, a method of using a three-dimensional (3D) cell-containing matrix comprises: providing a media chamber comprising a medium comprising (i) a plurality of cells and (ii) one or more polymer precursors. Next, the method may comprise directing at least one energy beam to the medium in the media chamber along at least one energy beam path that is patterned into a three-dimensional (3D) projection in accordance with computer instructions for printing the 3D cell-containing medical device in computer memory, to form at least a portion of the 3D cell-containing matrix comprising (i) at least a subset of the plurality of cells, and (ii) a polymer formed from the one or more polymer precursors. Next, the method may comprise positioning the 3D cell-containing matrix in a subject.

In another aspect, a method of using a three-dimensional (3D) cell-containing matrix, comprises (i) printing the 3D cell-containing matrix comprising a plurality of cells, and (ii) positioning the 3D cell-containing matrix in a subject.

In another aspect, a method for using a three-dimensional (3D) cell-containing matrix, comprises providing a media chamber comprising a first medium. The first medium may comprise a first plurality of cells and a first polymeric precursor. Next, the method may comprise directing at least one energy beam to the first medium in the media chamber along at least one energy beam path in accordance with computer instructions for printing the 3D cell-containing matrix in computer memory, to subject at least a portion of the first medium in the media chamber to form a first portion of the 3D cell-containing matrix. Next, the method may comprise providing a second medium in the media chamber. The second medium may comprise a second plurality of cells and a second polymeric precursor. The second plurality of cells may be of a different type than the first plurality of cells. Next, the method may comprise directing at least one energy beam to the second medium in the media chamber along at least one energy beam path in accordance with the computer instructions, to subject at least a portion of the second medium in the media chamber to form a second portion of the 3D cell-containing matrix. Next, the method may comprise positioning the first and second portions of the 3D cell-containing matrix in a subject.

In another aspect, a method of using a three-dimensional (3D) cell-containing matrix, comprises (i) printing the 3D cell-containing matrix comprising a first plurality of cells and a second plurality of cells. The first plurality of cells may be different from the second plurality of cells. Next, the method may comprise (ii) positioning the 3D cell-containing matrix in a subject.

The plurality of cells may be from a subject. The method plurality of cells may be selected from the list consisting of stromal endothelial cells, endothelial cells, follicular reticular cells or precursors thereof, naïve B cells or other immature B cells, memory B cells, plasma B cells, helper T cells and subsets of the same, effector T cells and subsets of the same CD+8 T cells, CD4+ T cells, regulatory T cells, natural killer T cells, naïve T cells or other immature T cells, dendritic cells and subsets of the same, follicular dendritic cells, Langerhans dendritic cells, dermally-derived dendritic cells, dendritic cell precursors, monocyte-derived dendritic cells, monocytes and subsets of the same macrophages and subsets of the same, leukocytes and subsets of the same. The B cells may be selected from the list consisting of naïve B cells, mature B cells, plasma B cells, B1 B cells and B2 B cells. The T cells may be selected from the list consisting of CD8+ and CD4+. The 3D cell-containing matrix may form a suture, stent, staple, clip, strand, patch, graft, sheet, tube, pin, or screws. The graft may be selected from the list consisting of skin implant, uterine lining, neural tissue implant, bladder wall, intestinal tissue, esophageal lining, stomach lining, hair follicle embed skin, and retina tissue.

The 3D cell-containing matrix may be from about 1 micrometer (μm) to about 10 centimeters (cm). The 3D cell-containing matrix may be from at least about 5 μm to about 10 cm or more. The 3D cell-containing matrix may be from at least about 10 μm to about 10 cm or more. The 3D cell-containing matrix may be from at least about 100 μm to about 10 cm or more. The 3D cell-containing matrix may be from at least about 500 μm to about 10 cm or more. The 3D cell-containing matrix may be from at least about 1000 μm to about 10 cm or more. The 3D cell-containing matrix may be from at least about 1 cm to about 10 cm or more. The 3D cell-containing matrix may be from about at least 5 to about 10 cm or more.

The 3D cell-containing matrix may be about 1 μm to about 1,000 μm. The 3D cell-containing matrix may be at least about 1 μm. The 3D cell-containing matrix may be at most about 1,000 μm. The 3D cell-containing matrix may be about 1 μm to about 5 μm, about 1 μm to about 10 μm, about 1 μm to about 100 μm, about 1 μm to about 1,000 μm, about 5 μm to about 10 μm, about 5 μm to about 100 μm, about 5 μm to about 1,000 μm, about 10 μm to about 100 μm, about 10 μm to about 1,000 μm, or about 100 μm to about 1,000 μm. The 3D cell-containing matrix may be about 1 μm, about 5 μm, about 10 μm, about 100 μm, or about 1,000 μm.

The 3D cell-containing matrix may be about 0.5 cm to about 10 cm. The 3D cell-containing matrix may be at least about 0.5 cm. The 3D cell-containing matrix may be at most about 10 cm. The 3D cell-containing matrix may be about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 9 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 9 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 4 cm to about 5 cm, about 4 cm to about 6 cm, about 4 cm to about 7 cm, about 4 cm to about 8 cm, about 4 cm to about 9 cm, about 4 cm to about 10 cm, about 5 cm to about 6 cm, about 5 cm to about 7 cm, about 5 cm to about 8 cm, about 5 cm to about 9 cm, about 5 cm to about 10 cm, about 6 cm to about 7 cm, about 6 cm to about 8 cm, about 6 cm to about 9 cm, about 6 cm to about 10 cm, about 7 cm to about 8 cm, about 7 cm to about 9 cm, about 7 cm to about 10 cm, about 8 cm to about 9 cm, about 8 cm to about 10 cm, or about 9 cm to about 10 cm. The 3D cell-containing matrix may be about 0.5 cm, about 1 cm, about 2 cm, about 3 cm, about 4 cm, about 5 cm, about 6 cm, about 7 cm, about 8 cm, about 9 cm, or about 10 cm.

The 3D cell-containing matrix may be at least about 1 μm or more. The 3D cell-containing matrix may be at least about 5 μm or more. The 3D cell-containing matrix may be at least about 10 μm or more. The 3D cell-containing matrix may be at least about 50 μm or more. The 3D cell-containing matrix may be at least about 100 μm or more. The 3D cell-containing matrix may be at least about 1000 μm or more. The 3D cell-containing matrix may be at least about 0.5 cm or more. The 3D cell-containing matrix may be at least about 1 cm or more. The 3D cell-containing matrix may be at least about 5 cm or more. The 3D cell-containing matrix may be at least about 10 cm or more.

The 3D cell-containing matrix may comprise an agent to promote growth of vasculature or nerves. The agent may be selected from the group consisting of growth factors, cytokines, chemokines, antibiotics, anticoagulants, anti-inflammatory agents, opioid pain-relieving agents, non-opioid pain-relieving agents, immune-suppressing agents, immune-inducing agents, monoclonal antibodies and stem cell proliferating agents.

Another aspect of the present disclosure provides a system for producing one or more immunological proteins, comprising a media chamber configured to contain a first medium comprising a first plurality of cells and a first plurality of polymer precursors. The system may comprise at least one energy source configured to direct at least one energy beam to the media chamber. The system may comprise one or more computer processors operatively coupled to the at least one energy source. The one or more computer processors may be individually or collectively programmed to receive computer instructions for printing a three-dimensional (3D) lymphoid organoid from computer memory. The one or more computer processors may be individually or collectively programmed to direct the at least one energy source to direct the at least one energy beam to the first medium in the media chamber along at least one energy beam path in accordance with the computer instruction, to subject at least a portion of the first polymer precursors to form at least a portion of the 3D lymphoid organoid. The one or more computer processors may be individually or collectively programmed to direct the at least one energy source to direct the at least one energy beam to a second medium in the media chamber along at least one energy beam path in accordance with the computer instructions, to subject at least a portion of the second medium in the media chamber to form at least a second portion of the 3D lymphoid organoid. The second medium may comprise a second plurality of cells and a second plurality of polymeric precursors. The second plurality of cells may be of a different type than the first plurality of cell. The one or more computer processors may be individually or collectively programmed to subject the first and second portions of the 3D lymphoid organoid to conditions sufficient to stimulate production of the one or more immunological proteins. The one or more computer processors may be individually or collectively further programmed to extract the one or more immunological proteins from the first and second portions of the 3D lymphoid organoid.

Materials that may be used to print 3D cell-containing matrices or devices include degradable polymers, non-degradable polymers, biocompatible polymers, extracellular matrix components, bioabsorbable polymers, hydrogels, or any combination thereof. Non-limiting examples of bioasborbable polymers include polyesters, polyamino acids, polyanhydrides, polyorthoesters, polyurethanes, and polycarbonates. Non-limiting examples of biocompatible polymers include collagen, hyaluronic acid and other glycosaminoglycans, poly-dl-lactic-co-glycolic acid (PLGA), poly-l-lactic acid (PLLA), polyglycolic acid (PGA), alginate, gelatin, agar, or a combination thereof. The biocompatible polymer may comprise an extracellular matrix component. Non-limiting examples of extracellular matrix components may include proteoglycans such as heparan sulfate, chondroitin sulfate, and keratan sulfate, non-proteoglycan polysaccharide such as hyaluronic acid, collagen, and elastin, fibronectin, laminin, nidogen, or any combination thereof. These extracellular matrix components may be functionalized with acrylate, diacrylate, methacrylate, cinnamoyl, coumarin, thymine, or other side-group or chemically reactive moiety to facilitate cross-linking induced directly by multi-photon excitation or by multi-photon excitation of one or more chemical doping agents. In some cases, photopolymerizable macromers and/or photopolymerizable monomers may be used in conjunction with the extracellular matrix components to create cell-containing structures. Non-limiting examples of photopolymerizable macromers may include polyethylene glycol (PEG) acrylate derivatives, PEG methacrylate derivatives, and polyvinyl alcohol (PVA) derivatives. In some instances, collagen used to create cell containing structure may be fibrillar collagen such as type I, II, III, V, and XI collagen, facit collagen such as type IX, XII, and XIV collagen, short chain collagen such as type VIII and X collagen, basement membrane collagen such as type IV collagen, type VI collagen, type VII collagen, type XIII collagen, or any combination thereof.

The biocompatible polymer may comprise other polymerizable monomers that are synthesized and not native to mammalian tissues, comprising a hybrid of biologic and synthetic materials. The biocompatible polymer may comprise a photoinitiator. Non-limiting examples of photoinitiators may include azobisisobutyronitrile (AIBN), benzoin derivatives, benziketals, hydroxyalkylphenones, acetophenone derivatives, trimethylolpropane triacrylate (TPT), acryloyl chloride, benzoyl peroxide, camphorquinone, benzophenone, thioxanthones, and 2-hydroxy-1-[4-(hydroxyethoxy)phenyl]-2-methyl-1-propanone. Hydroxyalkylphenones may include 4-(2-hydroxyethylethoxy)-phenyl-(2-hydroxy-2-methyl propyl) ketone (Irgacure® 295), 1-hidroxycyclohexyl-1-phenyl ketone (Irgacure® 184) and 2,2-dimethoxy-2-phenylacetophenone (Irgacure® 651). Acetophenone derivatives may include 2,2-dimethoxy-2-phenylacetophenone (DMPA). Thioxanthones may include isopropyl thioxanthone.

### Methods of Printing Three Dimensional Structures

The present disclosure provides methods and systems of printing and using a three-dimensional (3D) object. A method for printing and using a three-dimensional object may comprise generating a 3D projection corresponding to a first part of the object within a medium comprising at least one polymer precursor while simultaneously generating at least one additional projection corresponding to at least one additional part of the object in the medium. The combination of the first projection and at least one additional projection may form the 3D object. The at least one additional projection may be at least about 1, 2, 3, 4, 5, 6, 7, 8, 9, 10, 11, 12, 13, 14, 15, 20, 25, 50, 75, 100, or more additional projections. The at least one additional part may be at least about 2, 3, 4, 5, 6, 7, 8, 9, 10, 11, 12, 13, 14, 15, 20, 25, 50, 75, 100, or more additional parts. The at least one additional projection may be at most about 100, 75, 50, 25, 20, 15, 14, 13, 12, 11, 10, 9, 8, 7, 6, 5, 4, 3, 2, or fewer additional projections. The at least one additional part may be at most about 100, 75, 50, 25, 20, 15, 14, 13, 12, 11, 10, 9, 8, 7, 6, 5, 4, 3, 2, or fewer additional parts. The first part may be the same as the at least one additional part. For example, a plurality of first parts can be formed. Alternatively, the first part may be different from the at least one additional part. For example, a first part corresponding to an overall shape of an organ can be formed, and a second part corresponding to vasculature of the organ can be formed. The first projection and the at least one additional projection may be initiated simultaneously in the same print volume. The first and second projections can coexist within a same print volume. For example, a first projection can cure rough features of the object while the second projection can cure fine details of the object. The first projection may be initiated prior to the second projection. For example, the first projection can be initiated, and the second projection can be subsequently initiated. In another example, the first projection can be completed prior to the initiation of the second projection.

The medium may comprise two or more polymer precursors. The medium may comprise at least about 2, 3, 4, 5, 6, 7, 8, 9, 10, 11, 12, 13, 14, 15, 20, 25, 50, 75, 100, or more polymer precursors. The medium may comprise at most about 100, 75, 50, 25, 20, 15, 14, 13, 12, 11, 10, 9, 8, 7, 6, 5, 4, 3, 2, or fewer polymer precursors. Each polymer precursor of the two or more polymer precursors can be polymer precursors as described elsewhere herein. The two or more polymer precursors may be polymerizable under exposure to different energies. For example, one polymer may polymerize when exposed to 700 nm light while another polymer may polymerize when exposed to 400 nm light. In this example, the first projection may polymerize the 400 nm polymer while a second projection would polymerize the 700 nm polymer. The two or more polymer precursors may be polymerizable under different reaction conditions. For example, a first polymer can be polymerizable at 50° C. while a second polymer precursor can be polymerizable at 75° C. In another example, a first polymer precursor can be polymerizable in an absence of a cell product, while a second polymer precursor can be polymerizable in the presence of the cell product. In this example, the first projection can polymerize the first precursor, while the second projection can polymerize the second precursor in proximity to cells that generate the cell product.

The first projection may be a 3D projection, as described elsewhere herein. The first projection may be a holographic projection. For example, the first projection can be a projection of a plurality of points of light. The first projection may simultaneously or substantially simultaneously generate points in the x, y, and/or z axes (e.g., generate points through a volume). The first projection may be generated using methods and systems described elsewhere herein. The at least one additional projection may be a 3D projection. The at least one additional projection may be a holographic projection. The at least one additional projection may simultaneously or substantially simultaneously generate points in the x, y, and z axes. Alternatively, the at least one additional projection may be a 2D projection. The at least one additional projection may be generated using methods and systems described elsewhere herein.

The first projection may be generated using at least one phase and/or amplitude modulator and the at least one additional projection may be generated using the at least one phase and/or amplitude modulator or at least one other phase and/or amplitude modulator. In some cases, the first projection may be generated using at least one digital micromirror device (DMD) and the at least one additional projection may be generated using the at least one DMD or at least one other DMD. Alternatively, the first projection may be generated using at least one spatial light modulator (SLM) and the at least one additional projection may be generated using at least one DMD or at least one other DMD. As another alternative, the first projection may be generated using at least one SLM and the at least one additional projection may be generated using the at least one SLM or at least one other SLM. As another alternative, the first projection may be generated using at least one DMD and the at least one additional projection may be generated using at least one SLM or at least one other SLM. The first projection and the at least one additional projection may form the same part or different parts of the object.

The at least one additional projection may be limited in two dimensions while printing in the entire volume of the third. For example, the x and y dimensions of the at least one additional projection may be defined (e.g., have a shape that corresponds to the object) while the object would be generated throughout the z dimension of the medium. In this example, if the x and y dimension were controlled to the shape of a circle, the resulting object would be a cylinder with a height equal to the depth of the medium. Alternatively, the at least one additional projection may be controlled such that it forms a projection of a defined shape within two dimensions and has a height in the third dimension of at most about 1,000 μm, 500 μm, 250 μm, 100 μm, 50 μm, 40 μm, 30 μm, 20 μm, 10 μm, or less. The at least one additional projection may be controlled such that it forms a projection of a defined shape within two dimensions and has a height in the third dimension of at least about 10 μm, 20 μm, 30 μm, 40 μm, 50 μm, 100 μm, 250 μm, 500 μm, 1,000 μm, or more. For example, if the x and y dimensions were controlled to form a circle, and the z dimension were controlled to be a height of 50 μm, the resultant object can be a cylinder with a height of 50 μm. The at least one additional projection may be controlled in the third dimension by at least one SLM, at least one DMD, or any combination thereof.

The first projection may be a multi-photon (e.g., two-photon) projection. The first projection may be used to define fine features of the object. The fine features may be, but are not limited to, features of organs (e.g., thymic niches, alveoli), features of organoids, scaffolds, cellular niches, vasculature, microvasculature, a substrate for cellular growth, a grating, the outside bound of an object, or other features that benefit from high resolution. The fine features may have a feature size of at least about 10 nanometers (nm), 100 nm, 500 nm, 1 μm, 5 μm, 10 μm, 25 μm, 50 μm, 75 μm, 100 μm, 250 μm, 500 μm, 750 μm, 1,000 μm, 5,000 μm, 10,000 μm, or more. The fine features may have a feature size of at most about 10,000 μm, 5,000 μm, 1,000 μm, 750 μm, 500 μm, 250 μm, 100 μm, 75 μm, 50 μm, 25 μm, 10 μm, 5 μm, 1 μm, 500 nm, 100 nm, 10 nm, or less. The first projection may be used for multi-photon 3D printing. The multi-photon 3D printing may be as described elsewhere herein.

The at least one additional projection may be a single photon and/or a multi-photon (e.g., two-photon) projection. The at least one additional projection may be one projection or a plurality of projections. The at least one additional projection may be of the same energy as the first projection (e.g., having the same wavelength) or of a different energy as the first projection (e.g., having a different wavelength). In some cases, the light of the at least one additional projection may be of an energy about double that of the light of the first projection (e.g., the first projection may have an energy of 0.5 eV, while the second projection may have an energy of 1.0 eV). The at least one additional projection may comprise a plurality of projections, and a projection in the plurality of projections may have the same or different energies as other projections in the plurality of projections. For example, a first additional projection can have a wavelength of 700 nm while a second additional projection can have a wavelength of 1100 nm. The first projection and the second projection may be used to target different materials within the medium. For example, the first projection can cure a first material while the second projection can cure a second material. The at least one additional projection may be used for single-photon 3D printing (e.g., printing where a single photon carries sufficient energy to cure a portion of the medium).

The at least one additional projection may be used to cure and/or generate features larger than those generated by the first projection. In some cases, the first projection may be used to define the microvasculature of an organoid, while a second and third projection may be used to generate the rest of the structure of the organoid. In some cases, the first projection may be used to define the exterior of an object that may be used to form a mold for casting (where the resolution may impact the final detail and fidelity of the cast object), while the at least one additional projection may generate the non-surface bulk of the object. The combination of the first projection and the at least one additional projection simultaneously forming an object may result in faster production of the object while maintaining a high resolution.

The first projection and the at least one additional projection may be formed using the same optical elements (e.g., the same optical path, the same DMD, the same SLM, etc.). The first projection and the at least one additional projection may be formed on different optical elements. The different optical elements may be in parallel. The first projection and the at least one additional projection may occur simultaneously through the same optical objective. The first projection and the at least one additional projection may occur simultaneously different optical objectives. The first projection and the at least one additional projection may come from the same optical axis (e.g., the same side of the object) or different optical axes (e.g., different sides of the object).

The first projection and the at least one additional projection may occur simultaneously. The first projection and the at least one additional projection may occur substantially simultaneously. The first projection and the at least one additional projection may occur simultaneously for a time, after which the at least one additional projection may be stopped (e.g., by turning off the light source, by placing a shutter in the path of the light) and the first projection may continue after the at least one additional projection has been stopped. Stopping the at least one additional projection may allow for the first projection to deposit fine detail onto the object.

The first projection may be used to join objects generated by the one or more additional projections. The joining of the objects may be direct chemical bonding (e.g., forming chemical bonds between objects), “knitting” objects together by entangling non interacting polymers, or a combination thereof. The first projection may be able to penetrate into objects generated by the one or more additional projections. For example, the first projection may comprise light of a wavelength that the object is at least partially transparent to. The penetration may allow for the first projection to cure polymer precursors trapped within the object formed by the one or more additional projections. The one or more additional projections may generate an object containing one or more polymer precursors that are responsive to the light of the first projection. The one or more polymer precursors may be cured by the first projection, forming an object within the object generated by the one or more additional projections. The generation of an object within an object may join similar or dissimilar materials.

The methods and systems described herein may also be used for an ablative object generation process. The one or more additional projections may form the basis of the object. The first projection may be used to remove or ablate material from the object, thus forming fine features on or within the object. The first projection may be used to form the fine features by ablation while the one or more additional projections are printing other parts of the object. For example, the one or more additional projections can form a portion of the object, the first projection can begin forming fine detail in the first object by ablation, and the one or more additional projections can generate additional portions of the object. The first projection and the one or more additional projections may be used to simultaneously generate and ablate portions of the object. The first projection and the one or more additional projections may be concentrated in portions of the object to be ablated and left at lower powers in portions of the object to be generated. For example, both the first and the one or more additional projections can be used to ablate and/or form at least a portion of the object. The simultaneous generation and ablation may allow for seamless creation of positive and negative spaces on or within the object. The positive and negative spaces may be joined together as joints or connection systems.

The object formed by the first projection and the at least one additional projection may contain a cell or a plurality of cells. The cell or plurality of cells may be selected from a list of cells described herein. The plurality of cells may comprise one or more cell types. The cell or plurality of cells may replicate to impart functionality to the object (e.g., the cell may be liver cells and blood vessel cells that replicate to produce a functional liver). The cell may be of a subject. The subject may be a human, an animal, a microorganism, a plant, any of the aforementioned subjects suspected of having a disease, or any combination thereof. The cell may be a single celled organism. The object formed by the first projection and the at least one additional projection may not contain a cell or a plurality of cells. The formed by the first projection and the at least one additional projection may be configured to accept one or more cells after the object is formed. For example, the object can be printed in an absence of cells and have one or more cells introduced to the object after the printing.

The object may be printed based on computer instructions. The computer instructions may comprise a computer model of the object. The computer instructions may be based on an existing object. The object may be a substantially similar reproduction of an existing object. The computer instructions may be based off of the native structure of an organ, a 3D scan of an object, a point cloud 3D image formed of a plurality of 2D images, a magnetic resonance image scan, an ultrasound, a positron emission tomography scan, an x-ray computed tomography scan, an echocardiogram, or the like, or any combination thereof.

The object may be an organ or organoid as described herein (e.g., selected from a list of organs or organoids found herein). The object may be at least a part of an organ or organoid as described herein. The object may be formed for use in a subject. The object may be prepared for use in a subject. The preparation may comprise tissue culturing, incubation, introduction of fluids (e.g., blood, buffers, etc.), and other further processing steps. The object may be combined with another object. The other object may be another printed object, an organ of a subject, or another premade object.

Wavelengths may be used in numerous frequencies that may demonstrate benefits of local excitation or absorption between single wavelength energies, such as, for example one-photon, and dual combined wavelength energies, as for example two-photon. Many applications may benefit from using combined wavelengths, such as materials sciences, communications, manufacturing, and computing, with single and multi-wavelength absorption ranging from x-rays through radio waves.

### Computer Systems

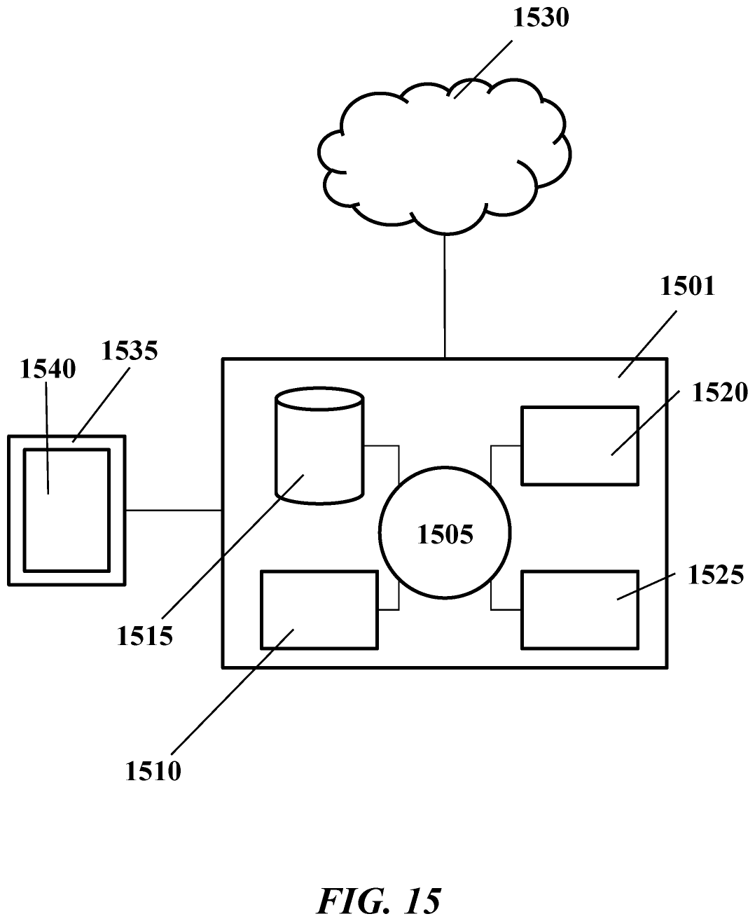

The present disclosure provides computer systems that are programmed to implement methods of the disclosure. FIG. 15 shows a computer system 1501 that is programmed or otherwise configured to implement the methods or control the systems of the present disclsoure. The computer system 1501 can regulate various aspects of the present disclosure, such as, for example, operation of a 3D printer. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (TR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, an interface for inputting a computer model to be printed by a 3D printer. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, process a 3D file for printing by a 3D printer.

The following examples are illustrative of certain systems and methods described herein and are not intended to be limiting

### Example 1—Printing a 3D Object Using Different Printing Parameters

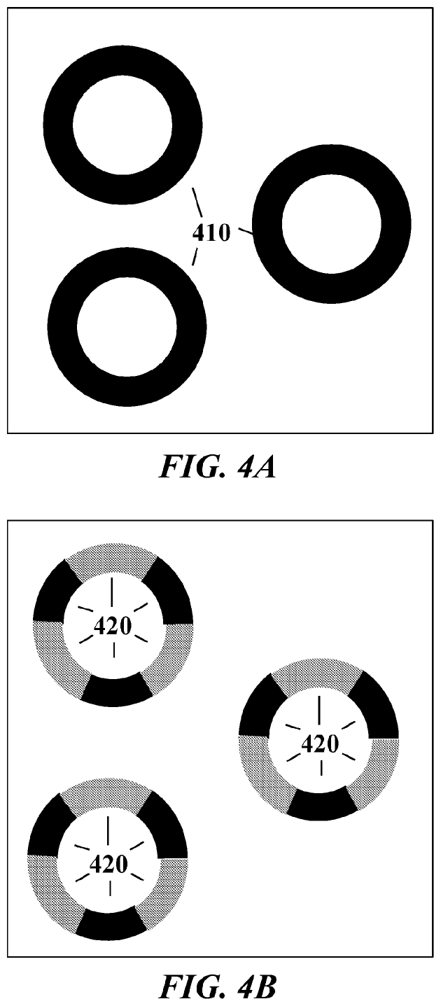

FIG. 3 is a microscope image of an example of a 3D object 300, according to some embodiments. The 3D object can comprise a plurality of different features, for example, vertical spokes 301, vertical vasculature 302, horizontal vasculature 303, intubation portion 304, or the like, or any combination thereof. The vertical spokes may be configured to provide structural support to the 3D object (e.g., the vertical spokes can be used to connect different portions of the 3D object to provide increased structural rigidity for those portions). The vertical and horizontal vasculature may be configured to provide a fluid (e.g., a nutrient fluid, blood, etc.) to the 3D object. The vasculature may enable growth of cells in the 3D object by providing a way to introduce nutrients to the cells and remove waste from the cells. The intubation port can be in fluidic communication with the vasculature. The intubation port may be configured as a inlet for the fluid to be provided to the 3D object. For example, the intubation port can be configured to receive fluid from an external pump and provide the fluid to the vasculature, and thereby the rest of the 3D object. The intubation port may be configured to interface with a capillary tube, a tube, a needle, a syringe, or the like, or any combination thereof.

Also visible in FIG. 3 may be a plurality of voxels. The boundaries of the voxels may appear as lines on the 3D object where the cure level of the overlap is minorly different from the cure level of the rest of the voxel. The voxels may be printed according to the methods described elsewhere herein.

### Example 2—Printed Microfluidic Platforms

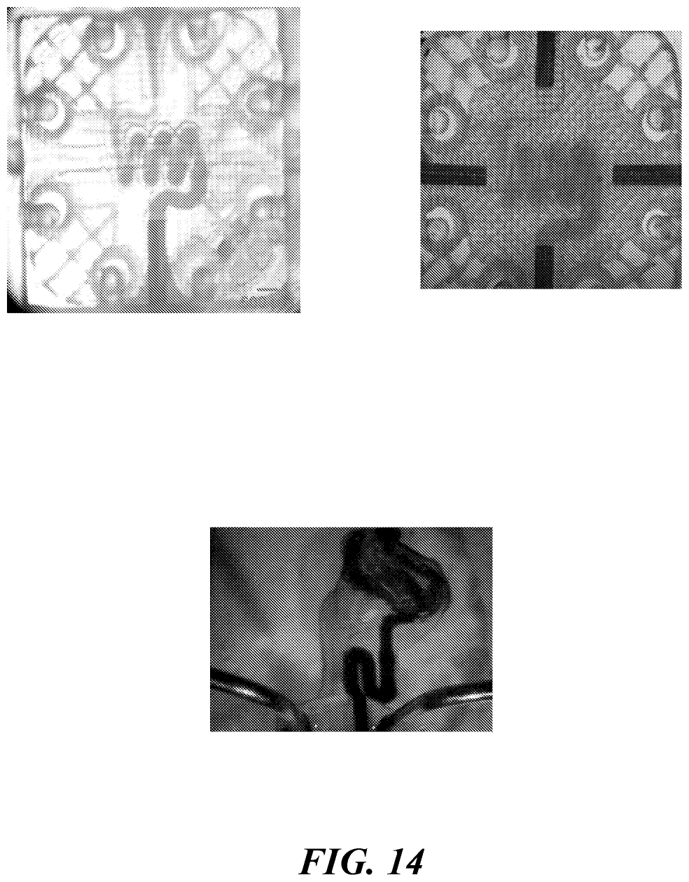

FIG. 14 is an example of microscope images of a microfluidics platform, according to some embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for printing a three-dimensional (3D) object, comprising:
(a) printing a first portion of said 3D object using a first parameter set and a first light beam, wherein said first parameter set includes at least one first parameter corresponding to a first optical property of said first light beam; and
(b) printing a second portion of said 3D object different from said first portion using a second parameter set and a second light beam, wherein said second parameter set includes at least one second parameter corresponding to a second optical property of said second light beam, wherein said second parameter set is different from said first parameter set, wherein said second optical property is different from said first optical property, to yield at least at least a portion of said 3D object comprising said first portion and said second portion.

2. The method of claim 1, wherein said first or second parameter set each comprise one or more parameters individually selected from the group consisting of voxel count, mod value, dwell time, illumination time, and optical power.

3. The method of claim 1, wherein said first portion and said second portion comprise different feature sizes.

4. The method of claim 1, wherein using said first parameter set and said second parameter set reduces an overprinting or an over-curing of said 3D object.

5. The method of claim 1, wherein the first light beam and the second light beam are both generated by a same light source.

6. The method of claim 5, wherein said light source is a laser light source.

7. The method of claim 1, further comprising printing a third portion of said 3D object different from said first portion or said second portion using a third parameter set and a third light beam.

8. The method of claim 7, wherein said third parameter set comprises a gradient of parameters between said first parameter set and said second parameter set.

9. The method of claim 1, further comprising printing a second 3D object configured to provide feedback on said printing said first portion and said printing said second portion.

10. The method of claim 1, wherein said first portion and said second portion have different properties.

11. The method of claim 10, wherein said properties are selected from the group consisting of feature size, tensile strength, porosity, Young's modulus, yield strength, degradation rate, swelling properties, protein composition, and polymer composition.

12. The method of claim 1, wherein said 3D object comprises one or more biopolymers.

13. The method of claim 1, wherein said using said second parameter set for said printing said second portion reduces an overcuring of said second portion as compared to using said first parameter set to print said second portion.

14. The method of claim 1, wherein said printing said 3D object comprises printing a plurality of portions to form said 3D object, wherein said plurality of portions comprises said first portion and said second portion.

15. The method of claim 1, wherein said first parameter set is configured to achieve a first predetermined level of cure of said first portion, and wherein said second parameter set is configured to achieve a second predetermined level of cure of said second portion.

16. The method of claim 1, wherein said second portion is at least partially disposed within said first portion, or vice versa

17. The method of claim 16, wherein said second portion is disposed within said first portion, or vice versa

18. The method of claim 1, wherein said 3D object is printed at a smaller size than a size where said 3D object will be used.

19. The method of claim 18, wherein said 3D object is printed 5% smaller than said size where said 3D object will be used.

20. The method of claim 18, wherein said 3D object is exposed to agents configured to swell said 3D object to said size where said 3D object will be used.

21. The method of claim 20, wherein said agents comprise phosphate buffered saline.

22. The method of claim 1, wherein said first portion and said second portion are printed at a substantially same time.

23. The method of claim 1, wherein said 3D object is printed in a time period of at most about 6 hours.

24. The method of claim 1, wherein said 3D object comprises at least one cell.

25. The method of claim 24, wherein said at least one cell is of a subject.

26. The method of claim 24, wherein said at least one cell is present in a media chamber prior to said directing.

27. The method of claim 24, wherein said at least one cell is introduced to said 3D object subsequent to generating said object.

28. The method of claim 1, wherein said first light beam comprises a holographic projection of said first portion or said second portion.

29. The method of claim 1, wherein said light beam comprises a plurality of energy beams.

30. The method of claim 1, wherein said 3D object corresponds to an organ or organoid selected from the group consisting of a two-dimensional organ or organoid, a three-dimensional organ or organoid, a lymph node, an islet of Langerhans, a hair follicle, a tumor or a tumor spheroid, a neural bundle and support cell(s), a nephron, a liver organoid, an intestinal crypt, a primary lymphoid organ, a secondary lymphoid organ, a spleen, a liver, a pancreas, a gallbladder, an appendix, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and a blood vessel.

31. The method of claim 1, further comprising receiving computer instructions for printing said 3D object, and forming at least said first portion or said second portion based at least in part on said computer instructions.

32. The method of claim 31, wherein said computer instructions comprise a computer model of said object.

33. The method of claim 1, wherein said 3D object comprises a polymeric material, a metal, a metal alloy, a composite material, or any combination thereof.

34. The method of claim 1, wherein said light beam is phase modulated.

35. The method of claim 1, wherein said 3D object comprises signaling molecules or proteins.

36. The method of claim 1, further comprising, subsequent to (a), developing said 3D object into a biologically functional tissue.

37. The method of claim 1, wherein said light beam is generated by least one laser source.

38. The method of claim 37, wherein said laser source is a two-photon energy source.

39. A method of generating a computer file corresponding to a three-dimensional (3D) object, wherein said computer file is usable for printing said 3D object using a three-dimensional (3D) printer, said method comprising:
(a) receiving a computer model of said 3D object into computer memory;
(b) slicing said computer model to form a plurality of voxels;
(c) distributing said plurality of voxels into a plurality of constellations, wherein a constellation of said plurality of constellations comprises at least one voxel of said plurality of voxels, wherein said constellation of said plurality of constellations and another constellation of said plurality of constellations are curable with an approximately same optical power; and
(d) generating said computer file comprising said plurality of constellations.

40. The method of claim 39, wherein said plurality of voxels are oriented in three dimensions relative to one another.

41. A method of preparing a file corresponding to a three-dimensional (3D) object for printing using a three-dimensional (3D) printer, comprising:
(a) receiving a plurality of clusters generated by a k-means fracturing algorithm; and
(b) recombining said plurality of clusters by maximizing a centroid distance for each cluster of said plurality of clusters.

42. A method of printing a three-dimensional (3D) object using a three-dimensional (3D) printer, comprising:
(a) using said 3D printer to cure a first portion of said 3D object; and
(b) using said 3D printer to cure a second portion of said 3D object, wherein said first portion and said second portion form an at least partially overlapping area, and wherein said at least partially overlapping area has a substantially same level of cure as said first portion and said second portion.

43. A method of printing a three-dimensional (3D) object using a three-dimensional (3D) printer, comprising:
(a) using said 3D printer to provide a first patterned light field to cure a first portion of said 3D object; and
(b) using said 3D printer to provide a second patterned light field to cure a second portion of said 3D object at least partially overlapping with said first portion of said 3D object to form an at least partially overlapping portion, wherein said first patterned light field and said second patterned light field comprise a region of lower light intensity within said at least partially overlapping portion.

44. A method of troubleshooting a three-dimensional (3D) printing process using a 3D printer, comprising:
(a) using said 3D printer to print a first object;
(b) using said 3D printer to print a second object, wherein said second object comprises a circle with an equilateral cross disposed therein; and
(c) comparing said second object with a computer file for said second object to troubleshoot said 3D printing process.

45. The method of claim 44, wherein said first object or said second object is printed in a time period of at most about 6 hours.

46. The method of claim 44, wherein said first object and said second object are printed at a substantially same time.

47. The method of claim 44, wherein said first object comprises at least one cell.

48. The method of claim 47, wherein said at least one cell is of a subject.

49. The method of claim 47, wherein said at least one cell is present in said media chamber prior to said printing.

50. The method of claim 47, wherein said at least one cell is introduced to said object subsequent to said printing.

51. The method of claim 44, wherein said printing comprises directing a three-dimensional holographic projection of at least one energy beam into a media chamber.

52. The method of claim 44, wherein said first object corresponds to an organ or organoid selected from the group consisting of a two-dimensional organ or organoid, a three-dimensional organ or organoid, a lymph node, an islet of Langerhans, a hair follicle, a tumor or a tumor spheroid, a neural bundle and support cell(s), a nephron, a liver organoid, an intestinal crypt, a primary lymphoid organ, a secondary lymphoid organ, a spleen, a liver, a pancreas, a gallbladder, an appendix, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and a blood vessel.

53. The method of claim 44, further comprising receiving computer instructions for printing said first object or said second object, and forming at least said portion of said first object or said second object based at least in part on said computer instructions.

54. The method of claim 53, wherein said computer instructions comprise a computer model of said first object or said second object.

55. The method of claim 44, wherein said first object or said second object comprises a polymeric material, a metal, a metal alloy, a composite material, or any combination thereof.

56. The method of claim 44, wherein said first object comprises signaling molecules or proteins.

57. The method of claim 44, further comprising, subsequent to (a), developing said first object into a biologically functional tissue.

58. A method of troubleshooting a three-dimensional (3D) printing process using a 3D printer, comprising:
(a) using said 3D printer to print a first object;
(b) using said 3D printer to print a second object, wherein said second object comprises a plurality of cross-hatched lattices and wherein a distance between said lattices is asymmetrical; and
(c) comparing said second object with a computer file for said second object to troubleshoot said 3D printing process

59. The method of claim 58, wherein said first object and said second object are printed at a substantially same time.

60. The method of claim 58, wherein said first object or said second object is printed in a time period of at most about 6 hours.

61. The method of claim 58, wherein said first object or said second object comprises at least one cell.

62. The method of claim 61, wherein said at least one cell is of a subject.

63. The method of claim 61, wherein said at least one cell is present in prior to said printing.

64. The method of claim 61, wherein said at least one cell is introduced to said first object or said second object subsequent to generating said first object or said second object.

65. The method of claim 58, wherein said printing comprises directing a three-dimensional holographic projection of at least one energy beam into a media chamber.

66. The method of claim 58, wherein said first object corresponds to an organ or organoid selected from the group consisting of a two-dimensional organ or organoid, a three-dimensional organ or organoid, a lymph node, an islet of Langerhans, a hair follicle, a tumor or a tumor spheroid, a neural bundle and support cell(s), a nephron, a liver organoid, an intestinal crypt, a primary lymphoid organ, a secondary lymphoid organ, a spleen, a liver, a pancreas, a gallbladder, an appendix, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and a blood vessel.

67. The method of claim 58, further comprising receiving computer instructions for printing said first object or said second object, and forming at least said portion of said first object or said second object based at least in part on said computer instructions.

68. The method of claim 67, wherein said computer instructions comprise a computer model of said first object or said second object.

69. The method of claim 58, wherein said first object or said second object comprises a polymeric material, a metal, a metal alloy, a composite material, or any combination thereof.

70. The method of claim 58, wherein said first object comprises signaling molecules or proteins.

71. The method of claim 58, further comprising, subsequent to (a), developing said first object into a biologically functional tissue.
